(19) United States
(12) Patent Application Publication
(10) Pub. No.: US 2007/0080628 A1
(43) Pub. Date: Apr. 12, 2007

(54) Electroluminescent devices comprising 2-(p-triphenyl)-3-phenyl-pyrazine derivatives

(76) Inventors: Thomas Schäfer, Basel (CH); Marine Peuchmaur, Apprieu (FR); Jonathan Rogers, White Plains, NY (US); Michael Robert Craig, New York, NY (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/578,981

(22) PCT Filed: Nov. 17, 2004
(86) PCT No.: PCT/EP04/52984

§ 371 Date: May 11, 2006

(30) Foreign Application Priority Data
Nov. 26, 2003 (EP) 03104389.6

(51) Int. Cl. C07D 241/02; H01J 1/62; H01J 63/04

(52) U.S. Cl. 313/504; 544/336; 257/E51.02

(57) ABSTRACT

Disclosed are electroluminescent devices that comprise organic layers that contain pyrazine compounds. The pyrazine compounds are suitable components of blue-emitting, durable, organo-electroluminescent layers. The electroluminescent devices may be employed for full color display panels in, for example, mobile phones, televisions and personal computer screens. Accordingly, the present invention relates to pyrazine compounds of formula (I), wherein X¹ is a group of formula (II) , or a C₁₆-C₃₀aryl group, which can optionally be substituted by E, X² is an aryl group or a heteroaryl group, which can optionally be substituted, Y¹ and Y² are independently of each other a hydrogen atom, C₁-C₁₈alkyl, which is optionally interrupted by O, an aryl group or a heteroaryl group, which can optionally be substituted.

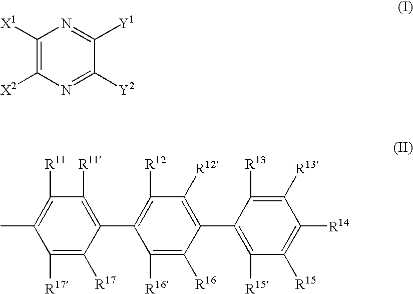

The present invention relates to organo-electroluminescent (EL) devices, in particular EL devices that comprise durable, blue-emitting organo-electroluminescent layers. The organo-electroluminescent layers comprise certain pyrazine compounds.

The present invention is aimed at an electroluminescent device comprising an organic light-emitting layer that contains at (east one blue-emitting pyrazine compound.

JP09188875A relates to a luminescent element comprising between an anode and a cathode a hole transport layer/electron transfer layer, a luminescent layer/electron transport layer or a monolayer structure made of a mixture of a luminescent material and an electron transport material and/or hole transporting material. An aromatic compound represented by the formula 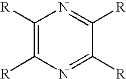
(wherein at least one R is an aromatic substituent and the number of nitrogen atoms of the substituents R is at least 1) having at least one six-membered ring structure and at least three nitrogen atoms in the molecule is vapor deposited on the electron transfer layer sandwiched between the anode and the cathode.

In U.S. Pat. No. 5,077,142 an organic EL device is described comprising 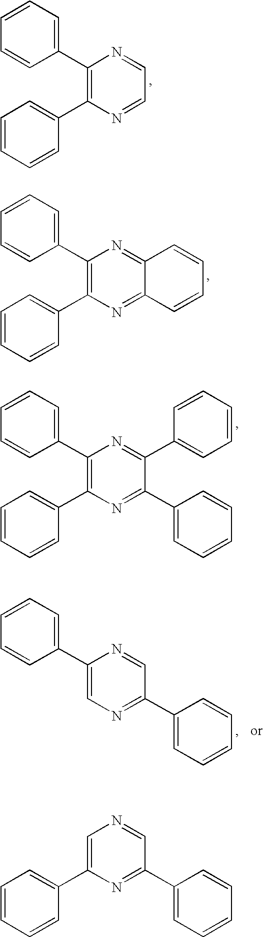
(see also JP06088072).
   JP08022040 discloses an organic non-linear optical material of formula 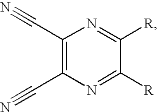
   wherein R is H, optionally substituted alkyl, alkoxy, optionally substituted aryl or aryloxy, optionally substituted alkylthio, alkylcarbonyloxy, alkylthiocarbonyloxy, OH, or halogen.

JP2003086381A, JP09188875A, JP2003040873A, JP1997188875A disdose EL devices, wherein quinoxaline compounds, such as 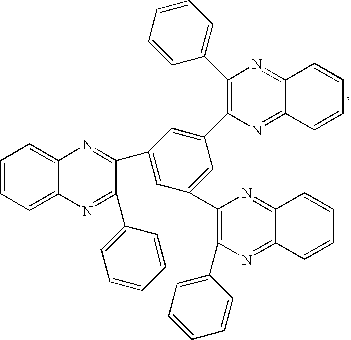
are used in the electron transport layer and/or electron injection layer.

JP2003109763A relates to EL devices, comprising the following pyrazine compound:

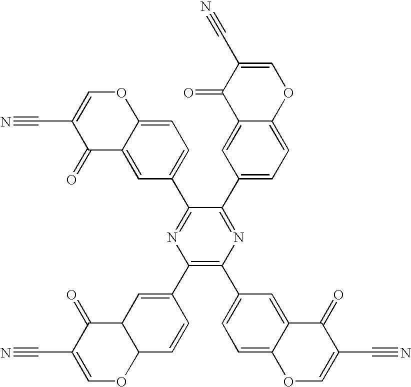

EP-A-763965 relates to blue emitting materials of formula 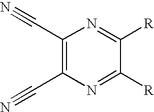
R=tert.-butyl phenoxyphenyl or methoxyphenyl and EL devices containing these materials.

EP-A-1148109 relates to EL devices, wherein among others quinoxaline compounds are used as host compounds.

WO02/088274 relates to EL devices, comprising double-spiro organic compounds, such as, for example, chemical compound 209:

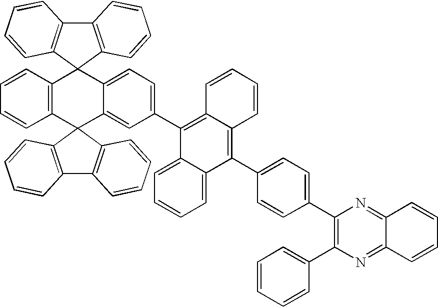

It is the object of the present invention to provide a light emitting element with excellent light emitting characteristics and durability.

Accordingly the present invention relates to an electroluminescent device, comprising a pyrazine compound of formula I.

In a preferred embodiment the electroluminescent device comprises in this order
(a) an anode
(b) a hole injecting layer and/or a hole transporting layer
(c) a light-emitting layer
(d) optionally an electron transporting layer and
(e) a cathode, wherein the pyrazine compound of formula I is preferably contained in the light-emitting layer.

In addition the present Invention is also directed to the use of the pyrazine compounds of formula I for electrophotographic photoreceptors, photoelectric converters, solar cells, image sensors, dye lasers and electroluminescent devices.

The pyrazine compounds of formula I are novel and form a further object of the present invention.

Accordingly, the present invention relates also to pyrazine compounds of formula

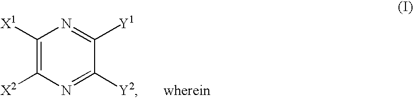

X¹ is a group of formula 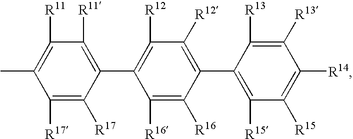
or a C₁₆-C₃₀aryl group, which can optionally be substituted by E;

X² is an aryl group, or a hetemaryl group, which can optionally be substituted; especially a group of formula RR or 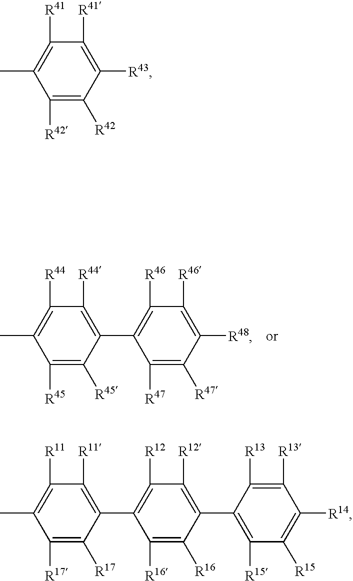
or a C₁₆-C₃₀aryl group, which can optionally be substituted by E;

Y¹ and Y² are independently of each other a hydrogen atom, C₁-C₁₈alkyl, which is optionally interrupted by O,

an aryl group or a heteroaryl group, which can optionally be substituted; especially a C₁₆-C30aryl group, which can optionally be substituted by E; or a group of formula

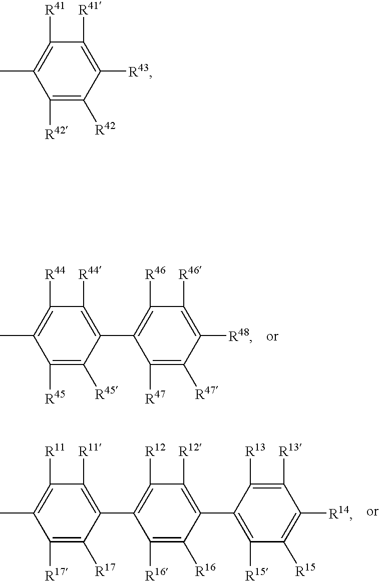

Y¹ and Y² together form a C₁-₈cydoalkyl group, wherein

R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by E; C₁-C₁₈alkyl; C₁-C₁₈alkyl which is substituted by E′ and/or interrupted by D;

C₇-C₁₈aralkyl; or C₇-C₁₈aralkyl which is substituted by E; or

R^{11′} and R¹², R^{12′} and R¹³, R^{15′} and R¹⁶, R^{16′} and R¹⁷, R^{44′} and R⁴⁶ and/or R^{45′} and R⁴⁷ are each a divalent group L¹ selected from an oxygen atom, an sulfur atom, >CR¹⁸R¹⁹>SiR¹⁸R¹⁹, or

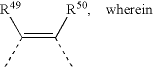

R¹⁸ and R¹⁹ are independently of each other C₁-C₁₈alkyl; C₁-C₁₈alkoxy, C₆-C₁₈aryl, C₁-C₁₈aryl, which is substituted by E; C₇-C18aralkyl, or C₇-C₁₈aralkyl, which is substituted by E; or

R¹¹ and R^{11′}, R¹² and R^{12′}, R¹³ and R^{13′}, R^{13′} and R¹⁴, R¹⁴ and R¹⁵, R¹⁵ and R^{15′}, R¹⁶ and R^{16′}, R¹⁷ and R¹⁷, R⁴¹ and R^{41′}, R⁴² and R^{42′}, R^{42′} and R⁴³, R⁴¹ and R^{43,} R⁴⁴ and R^{44′}, R⁴⁵ and R^{45′}, R⁴⁶ and R^{46′}, R⁴⁷ and R^{47′}, R^{46′} and R⁴⁸ and/or R^{47′} and R⁴⁸ are each a divalent group

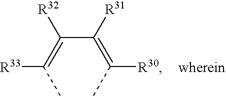

R³⁰, R³¹, R³², R³³, R⁴⁹ and R⁵⁰ are independently of each other H, C₁-C₁₈alkyl; C₁-C₁₈alkyl, which is substituted by E′ and/or interrupted by D; E; C₆-C₁₈aryl; C₆-C₁₈aryl, which is substituted by E;

R¹⁴ is H, C₂-C30heteroaryl, —NR⁷⁰R⁷¹, C₆-C₃₀aryl, or C₆-C₃₀aryl which is substituted by E, C₁-C₁₈alkyl; or C₁-C₁₈alkyl which is substituted by E′ and/or interrupted by D; especially

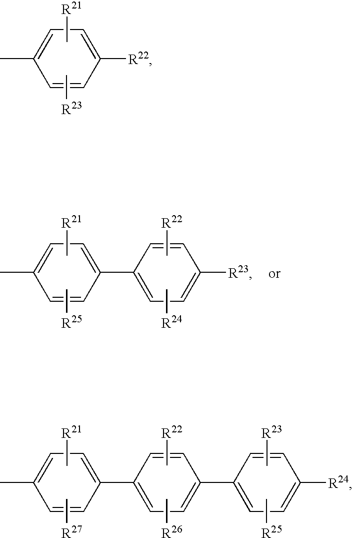

R²², R²³, R²⁴, R², R²⁶ and R²⁷ are independently of each other H, E, C₁-C₁₈alkyl; C₁-C₁₈alkyl which is substituted by E′ and/or interrupted by D; E; C7C₁₈aralkyl; C7C₁₈aralkyl which is substituted by E;

R⁴³ and R⁴⁸ are independently of each other H, E; especially C₁-C₂₄alkyl, C₁-C₂₄alkoxy, or —NR⁷⁰R⁷¹, wherein R⁷⁰ and R⁷¹ are independently of each other H, C₆-C₁₈aryl, C₆-C₁₈aryl which is substituted by C₁-C₂₄alkyl, or C₁-C₂₄alkoxy, C₁-C24alkyl, or C₁-C₂₄alkyl which is interrupted by —O—, or

R⁷⁰ and R⁷¹ together form a five or six membered ring, in particular 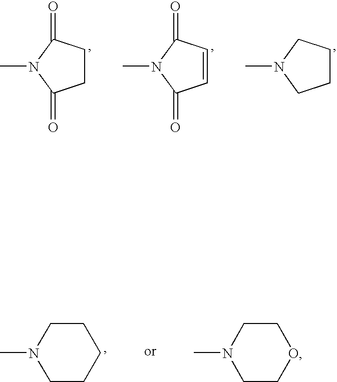
C₁-C₈alkyl; C₁-C₁₈alkyl, which is substituted by E and/or interrupted by D; C₂-C₃₀heteroaryl; C₇-C18aralkyl; C₇-C₁₈aralkyl which is substituted by E;

D is —CO—; —COO—; —OCOO—; —S—; —SO—; —SO₂—; —O—; —NR⁵—; —SiR⁶¹R⁶²—; —POR⁵—; —CR⁶³═CR⁶⁴—; or —C≡C—;

E is C₁-C₁₈alkyl, —OR⁵; —SR⁵; —NR⁵R⁶; —COR⁸; —COOR⁷; —CONR⁵R⁶; —CN; or halogen;

E′ is E, except C₁-C₁₈alkyl, wherein

R⁵ and R⁶ are independently of each other C₆-C₁₈aryl; C₆-C₁₈aryl which is substituted by C₁-C₁₈alkyl, or C₁-C₁₈alkoxy; C₁-C₁₈alkyl, or C₁-C₁₈alkyl which is interrupted by —O—; or

R⁵ and R⁶ together form a five or six membered ring, in particular

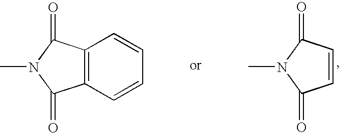

R⁷ is C₆-C₁₈aryl; C₆-C₁₈aryl which is substituted by C₁-C₁₈alkyl, or C₁-C₁₈alkoxy; C₁-C₁₈alkyl; or C₁-C₁₈alkyl which is interrupted by —O—;

R⁸ is C₇-C₁₂alkylaryl; C₁-C₁₈alkyl; or C₁-C₁₈alkyl which is interrupted by;

R⁶¹ and R⁶² are independently of each other C₆-C₁₈aryl; C₆-C₁₈aryl which is substituted by C₁-C₁₈alkyl, or C₁-C₁₈alkoxy; C₁-C₁₈alkyl, or C₁-C₁₈alkyl which is interrupted by —O—, and

R⁶³ and R⁶⁴ are independently of each other H, C₈-C₁₈aryl; C6-C₁₈aryl which is substituted by C₁-C₁₈alkyl, or C₁-C₁₈alkoxy; C₁-C₁₈alkyl, or C₁-C₁₈alkyl which is interrupted by —O—.

R¹¹, R_{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷, and R^{47′} as well as R¹⁴, R⁴³, and R⁴⁸ are preferably independently of each other H, E; or C₁-C₈alkyl, especially H, C₁-C₄alkyl, C₁-C₄alkoxy, or phenyl; wherein E is —OR⁵; —SR⁵; —NR⁵R⁶; —COR⁸; —COOR⁷; —CONR⁵R⁶; —CN; —OCOOR⁷; or halogen, especially F; wherein R⁵ and R⁶ are independently of each other C₆-C₁₂ary, or C₁-C₈alkyl;

R⁷ is C₇-C₁₂ alkylaryl, or C₁-C₈alkyl; and

R⁸ is C₆-C₁₂aryl; or C₁-C₈alkyl, or

R¹¹ and R^{11′}, R¹² and R^{12′}, R¹³ and R¹³, R^{13′} and R¹⁴, R⁴¹ and R^{41′}, R^{41′} and R⁴³, R⁴⁴ and R^{44′}, R⁴⁶ and R^{46′}, R^{46′} and R⁴⁸and/or R^{47′} and R⁴⁸ are each a divalent group

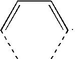

According to the present invention at least X¹, preferably X¹ and X² are a group of formula 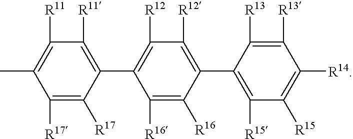
X¹ and X² can be different, but are preferably the same.

Preferably X¹ and X² are independently of each other a group of formula 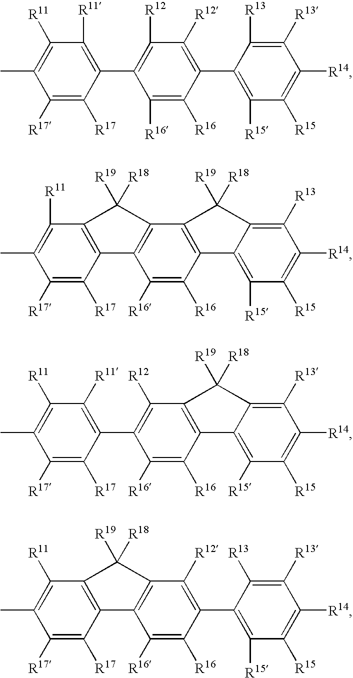
or —X¹¹—X¹²—X¹³, wherein R¹¹, R^{11′}, R¹², R^{12′}, R¹³, R^{13′}, R¹⁵, R^{15′}, R¹⁶, R16′, R¹⁷, and R^{17′} are independently of each other H, C₆-C₁₈aryl; C₆-C₁₈aryl which is substituted by E; E, C₁-C18alkyl; C₁-C₁₈alkyl which is substituted by E′ and/or interrupted by D; C₇-C₁₈aralkyl; C₇-C₁₈aralkyl which is substituted by E; and

R¹⁴, R¹⁸ and R¹⁹ are as defined above,

X¹¹ and X¹² are independently of each other a group of formula 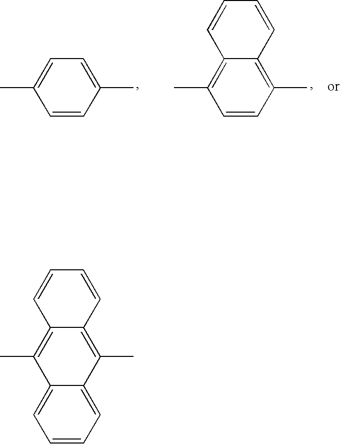
and X¹³ is a group of formula 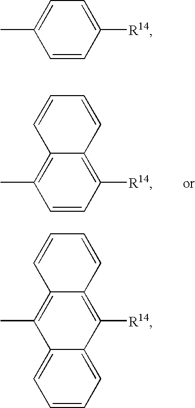
wherein R¹⁴ is 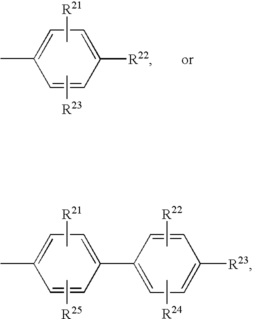
wherein R²¹, R²², R²³, R²⁴ and R²⁵ are as defined above and Y¹ and Y² are a hydrogen atom, C₁-C₁₈alkyl, which is optionally interrupted by O, or Y¹ and Y² together form a C₅-C₈cydoalkyl group, and are especially a hydrogen atom.

In a preferred embodiment X¹ and X² are a group of formula

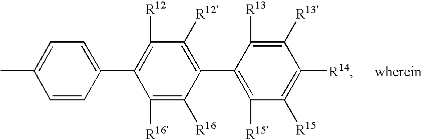

R¹³, R¹³, R¹⁵ and R^{15′} are H and R¹⁴ is H, or 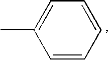
and R¹², R^{12′}, R¹⁶ and R^{16′} are H; or R¹³ and R¹⁵ are H, R^{13′} and R are independently of each other H, C₁-C₈alkyl, or C₁-C₈alkoxy, and R¹⁴ is H, C₁-C₈alkyl, or C₁-C₈alkoxy, and R¹², R^{12′}, R¹⁶ and R^{16′} are H, wherein at least one of R¹³, R¹⁵, R^{13′}, R^{15′} and R¹⁴ is C₁-C₈alkyl, or C₁-C₈alkoxy; or R¹² and R^{12′}, R¹³ and R^{13′}, R^{13′} and R¹⁴, R¹⁴ and R¹⁵, R¹⁵ and R^{15′}, and/or R¹⁶ and R^{16′}, are a divalent group

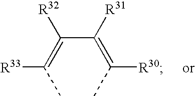

R^{12′}, R¹⁶, R^{16′} are H and R¹³ and R^{13′}, and/or R^{13′} and R¹⁴ are a divalent group

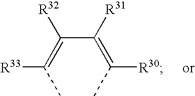

R¹³, R^{13′}, R¹⁴ , R¹⁵, R^{15′} are H and R¹² and R^{12′}, and/or R¹⁶ and R^{16′} are a divalent group 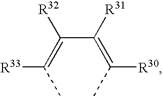
wherein R³⁰, R³¹, R³² and R³³ are H, C₁-C₈alkyl, or C₁-C₈alkoxy, and Y¹ and Y² are a hydrogen atom.

In a preferred embodiment X¹ and X² are independently of each other a group of formula 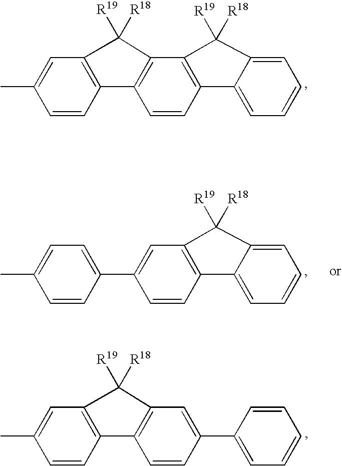
wherein R¹⁸ and R¹⁹ are independently of each other C₁-C₈alkyl.

In a preferred embodiment X¹ is a group of formula 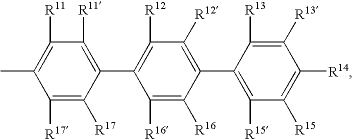
especially

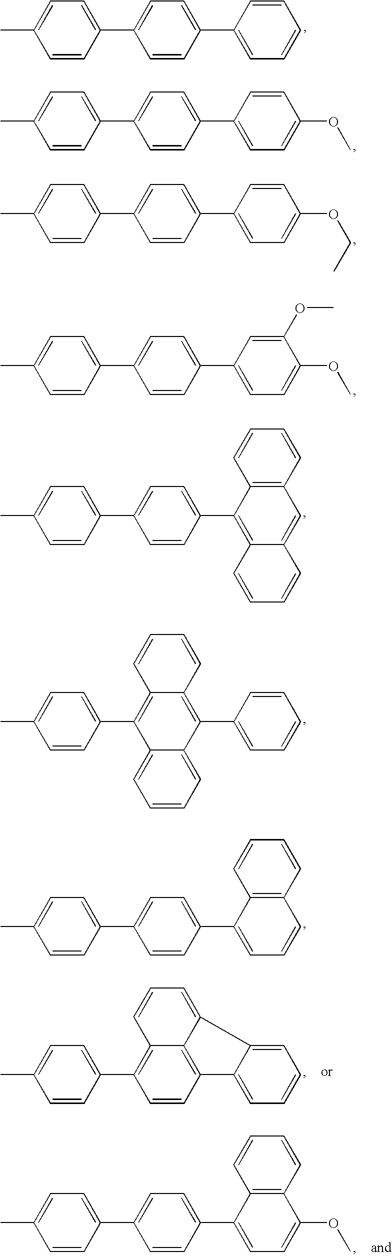

X² is a group of formula 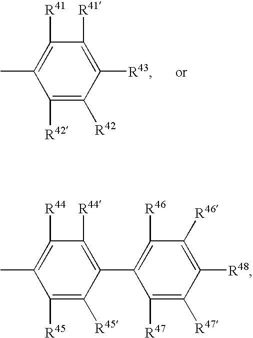
especially 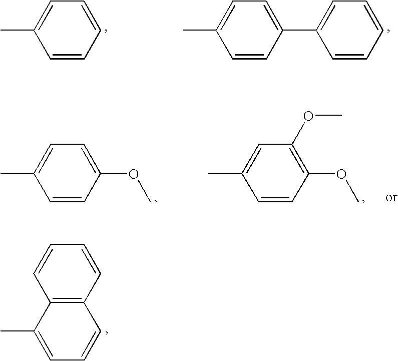
in particular a group of formula 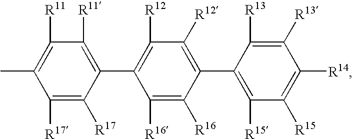
such as 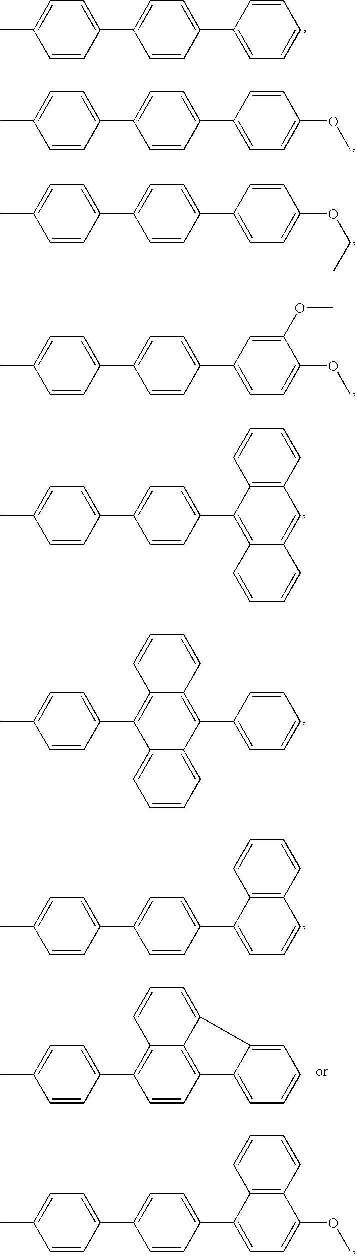
wherein R¹¹, R^{11′}, R¹², R^{12′}, R¹³, R^{13′}, R¹⁴, R¹⁵, R^{15′}, R¹⁶, R^{16′}, R¹⁷, R^{17′}, R⁴¹, R^{41′}, R⁴², R^{42′}, R⁴⁴, R⁴⁴, R⁴⁵, R^{45′}, R⁴⁶, R^{46′}, R⁴⁷, R^{47′, R}⁴³ and R⁴⁸ are as defined above and are especially H, C₁-C₈alkyl, C₁-C₈alkoxy, or phenyl, or

R¹³ and R^{13′}, R^{13′} and R¹⁴, R¹⁴ and R¹⁵, or R¹⁵ and R^{15′} can be a divalent group 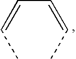
and Y¹ and Y² are a hydrogen atom.

R¹¹, R^{11′}, R¹², R^{12′}, R¹³, R^{13′}, R¹⁵, R^{15′}, R¹⁶, R^{16′}, R¹⁷ and R^{17′}, R⁴¹, R^{41′}, R⁴², R^{42′}, R⁴⁴, R^{44′}, R⁴⁵, R^{45′}, R⁴⁶, R^{46′}, R⁴⁷, and R^{47′} as well as R¹⁴, R⁴³, and R⁴⁸ are preferably independently of each other H, E; or C₁-C₈alkyl; wherein E is —OR⁵; —SR⁵; —NR⁵R⁸; —COR⁸; —COOR⁷; —CONR⁵R⁸; —CN; —OCOOR⁷; or halogen; wherein R⁵ and R⁶ are independently of each other C₆-C₁₂aryl, or C₁-C₈alkyl; R⁷ is C₇-C₁₂alkylaryl, or C₁-C₈alkyl; and R⁸ is C₆-C₁₂aryl; or C₁-C₈alkyl.

If X¹ and/or X² as well as Y¹ and/or Y² are a C₁₆-C₃₀aryl group, they are especially a fluoranthenyl, triphenlenyl, chrysenyl, naphthacen, picenyl, perylenyl, such as 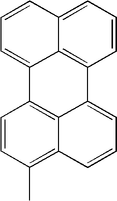
or 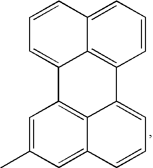
pentaphenyl, hexacenyl, or pyrenyl group, which can be substituted by E; very especially a fluoranthenyl group, which can be substituted by E.

Accordingly, in a further preferred embodiment the present invention is directed to compounds of formula I, wherein Y¹ and Y² are hydrogen and X¹ and X² are independently of each other a group Ar¹-Ar², wherein

Ar¹ is a group of formula

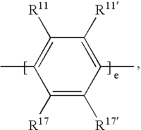

Ar² is a group of formula 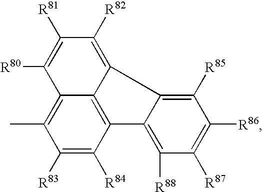
wherein

R⁸⁰, R⁸¹, R⁸² , R⁸³, R⁸⁴, R⁸⁵, R⁸⁶, R⁸⁷ and R⁸⁸ are independently of each other H, E′, C₆-C₁₈aryl; C₆-C₁₈aryl, which is substituted by E; C₁-C₁₈alkyl; C₁-C₁₈alkyl which is substituted by E′ and/or interrupted by D; C₇-C₁₈aralkyl; or C₇-C₁₈aralkyl which is substituted by E; e is an integer 1, or 2, and R¹¹, R^{11′}, R¹⁷ and R^{17′} are defined as above.

In said embodiment compounds of formula I are especially preferred, wherein X¹ and X² are a group Ar¹-Ar², wherein

Ar¹ is a group of formula

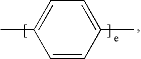

Ar² is a group of formula 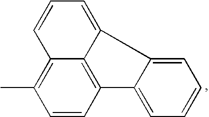
and e is an integer 1, or 2.

In a further preferred embodiment the present invention is directed to compounds of formula I, wherein Y¹ and Y² are independently of each other a group of the formula —W¹−(W²)_{b}—W³, wherein b is 0, or, 1, especially hydrogen, and X¹ and X² are independently of each other a group —W¹−(W²)_{b}—W³, wherein

W¹ and W² are independently of each other a group of formula

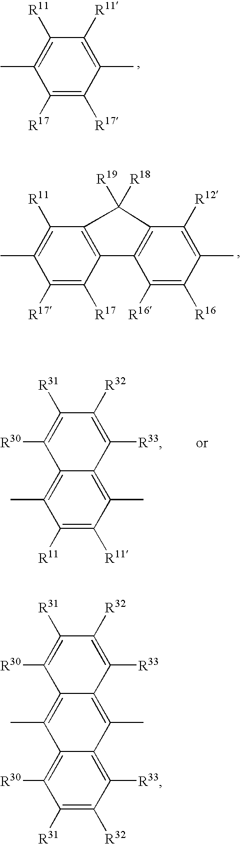

W³ is a group of formula 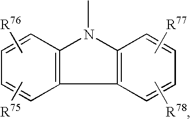
or —NR⁷⁰R⁷¹ , wherein R⁷⁰ and R⁷¹ are independently of each other a group of formula 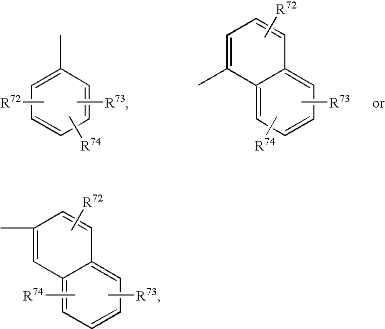
wherein R⁷², R⁷³ and R⁷⁴ are independently of each other hydrogen, C₁-C₈alkyl, C₁-C₈alkoxy, C₁-C₈alkylthio, a cyano group, a carbamoyl group, an amino group, a silyl group or a siloxanyl group,

R⁷⁵, R⁷⁶, R⁷⁷ and R⁷⁸ are independently of each other H, E, C₆-C₁₈aryl; C₆-C₁₈aryl which is substituted by C₁-C₁₈alkoxy, C₁-C₁₈alkyl, C₁-C₁₈alkyl which is interrupted by —O—; C₇-C₁₈aralkyl; or C₇-C₁₈aralkyl which is substituted by C₁-C₁₈alkoxy; wherein E, R¹¹, R^{11′}, R^{21′}, R¹⁶, R^{16′}, R¹⁷, R^{17′}, R¹⁸, R¹⁹, R³⁰, R³¹, R³² and R³³ are as defined above.

In said embodiment compounds of formula I are especially preferred, wherein Y¹ and Y² are hydrogen,

X¹ and X² are a group of the formula —W¹−(W²)_{b}—W³, wherein b is 0, or 1,

W¹ is a group of formula

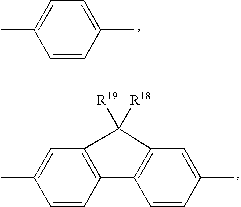

W² is a group of formula

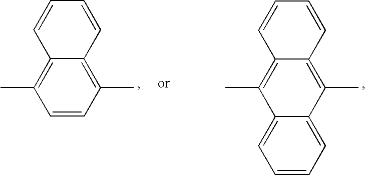

W³ is a group of formula 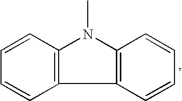
or —NR⁷⁰R⁷¹, wherein R⁷⁰ and R⁷¹ are independently of each other a group of formula 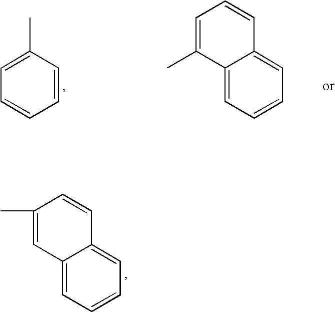
and R¹⁸ and R¹⁹ are independently of each other C₆-C₁₈alkyl.

In a particularly preferred embodiment of the present invention the pyrazine is a compound of formula I,

wherein X¹ is a group of formula 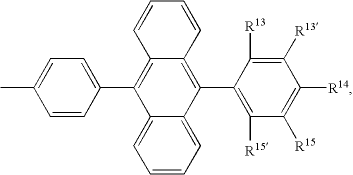
wherein R¹³, R¹³, R¹⁴, R¹⁵ and R^{15′} are independently of each other H, C₁-C₈alkyl, especially methyl, ethyl, n-butyl, t-butyl, C₁-C₁₈alkoxy, especially methoxy, ethoxy, butoxy, phenyl, phenoxy, or R¹³ and R^{13′} or

R^{13′} and R¹⁴ are a divalent group

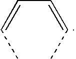

In a preferred embodiment of the present invention the pyrazine is a compound of formula I, wherein Y¹ and Y² are hydrogen. X¹ and X² can be different, but are preferably the same.

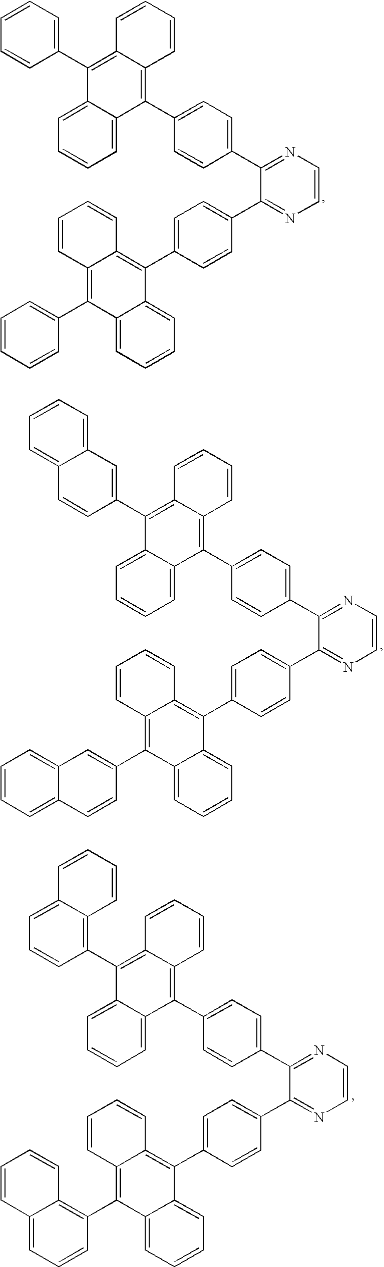

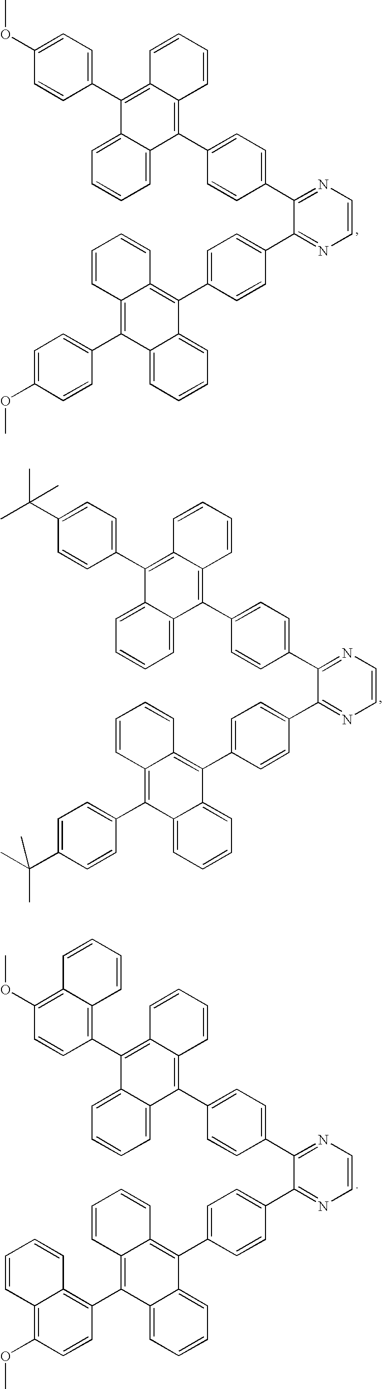

In a preferred embodiment of the present invention the pyrazine is a compound of formula I, wherein

X¹ is a group of formula 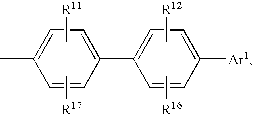
wherein Ar¹ is a group of formula

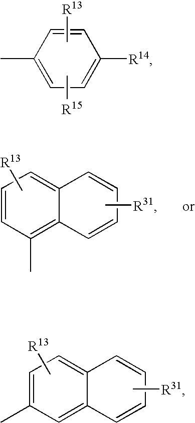

X² is a group of formula

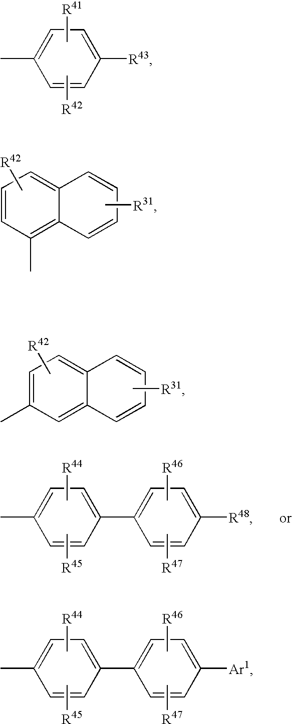

Y¹ and Y² are independently of each other H, C₁-C₈alkyl, or Ar2, wherein

R¹¹, R¹², R¹³, R¹⁵, R¹⁶, R¹⁷, R³¹, R⁴¹, R⁴², R⁴⁴, R⁴⁵, R⁴⁶ and R⁴⁷ are independently of each other H, -OR⁵, —NROR⁶, C₁-C₈alkyl, or phenyl,

R¹⁴ is H, —OR⁵, —NR⁶R_{6′}, or C₁-C₈alkyl,

R⁴³ and R⁴⁸ are independently of each other H, —OR⁵, —NR⁶R^{6′}, C₁-C₈alkyl, or phenyl, R⁵ is C₁-C₈alkyl, or phenyl, and

R⁶ and R^{6′} are independently of each other C₁-C₈alkyl.

Specific examples of preferred pyrazine compounds are given below:

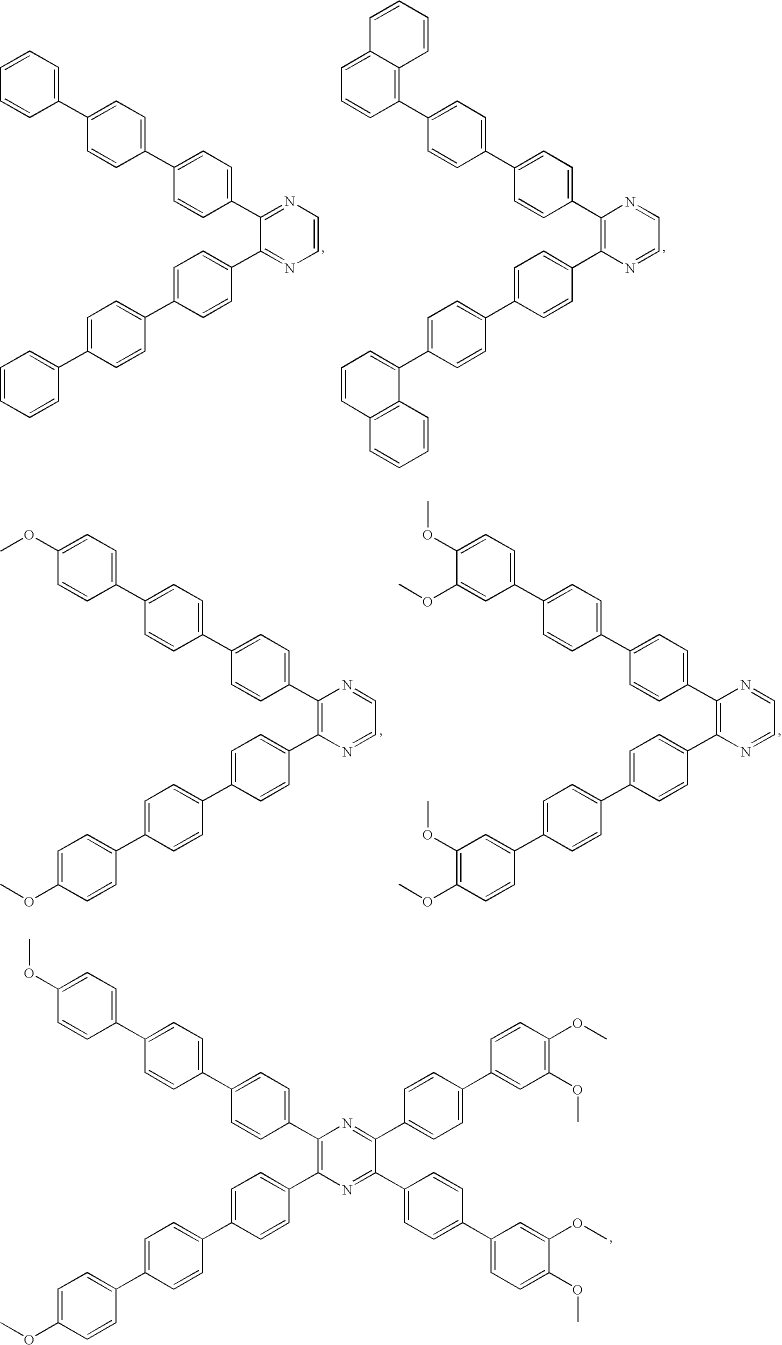

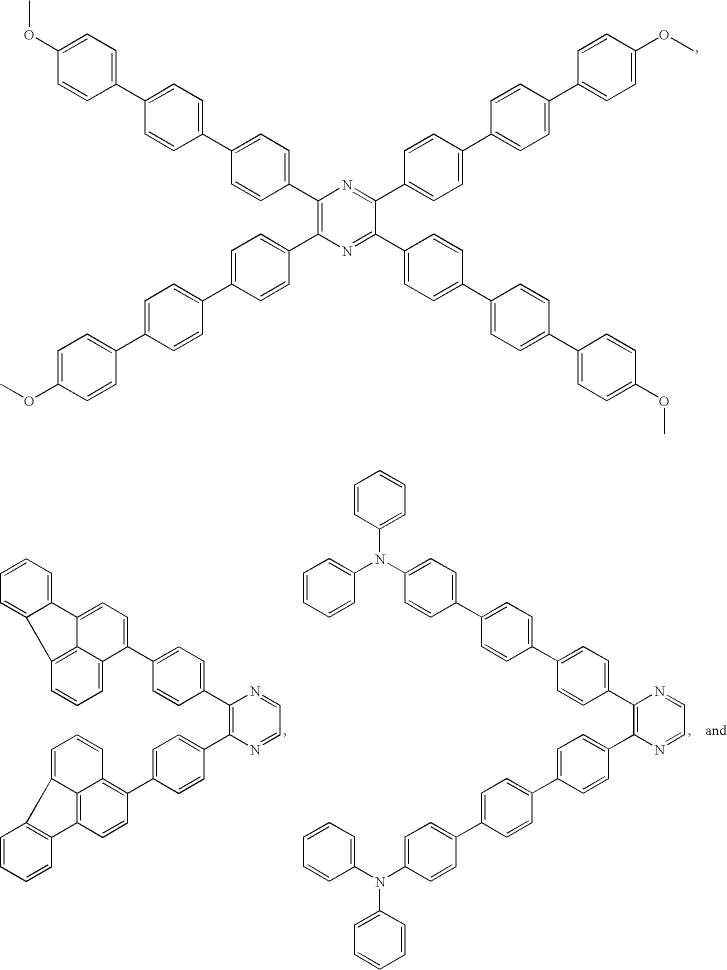

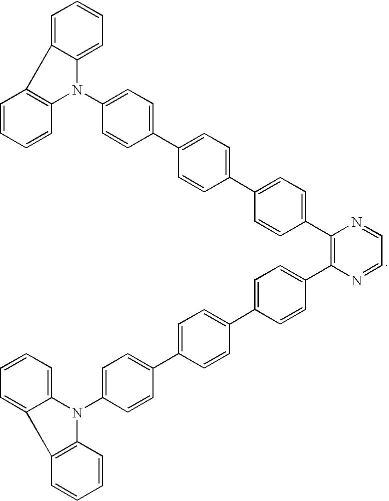

The present pyrazine compounds can be prepared according to or analogous to known procedures. The pyrazine compounds of the present invention of the formula: 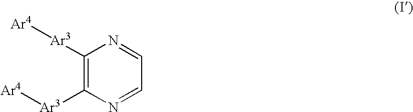
can, for example, be prepared according to a process (N. Miyaua and A. Suzuki in Chemical Reviews, Vol. 95, pp. 457-2483 (1995), which comprises reacting a derivative of formula 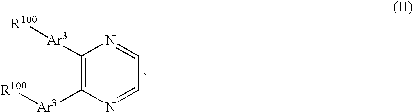
wherein R¹⁰⁰ stands for halogen such as chloro or bromo, preferably bromo, or E¹ having the meaning of 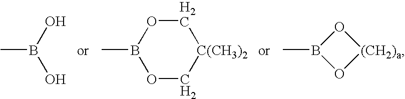
wherein a is 2 or 3,

with boronic acid derivative

E¹-Ar⁴,

or—in case R¹⁰⁰ is not halogen—Hal-Ar⁴,

wherein Hal stands for halogen, preferably for bromo,

wherein Ar³ is a group of formula 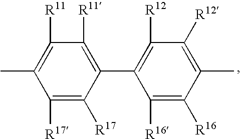
and Ar⁴ is a group of formula 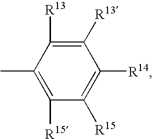
in the presence of a palladium catalyst, especially an allylpalladium catalyst of the μ-halo(triisopropylphosphine)(ηn³-allyl)palladium(II) type (see for example WO99/47474). The reaction is typically conducted at about 70° C. to 120° C. in an aromatic hydrocarbon solvent such as toluene. Other solvents such as dimethylformamide and tetrahydrofuran can also be used alone, or in mixtures with an aromatic hydrocarbon. An aqueous base, preferably sodium carbonate or bicarbonate, is used as the HBr scavenger. Depending on the reactivities of the reactants, a polymerization reaction may take 2 to 100 hours. Organic bases, such as, for example, tetraalkylammonium hydroxide, and phase transfer catalysts, such as, for example TBAB, can promote the activity of the boron (see, for example, Leadbeater & Marco; Angew. Chem. Int. Ed., 2003, 42, 1407 and references cited therein). Other variations of reaction conditions are given by T. I. Wallow and B. M. Novak in Journal of Organic Chemistry, Vol. 59, pp. 5034-5037 (1994); and M. Remmers, M. Schulze, and G. Wegner in Macromolecular Rapid Communications, Vol. 17, pp. 239252 (1996).

The compound of formula II can, for example, be obtained by reacting a compound of formula V and ethylene diamine and oxidizing the obtained compound of formula IV with DDQ.

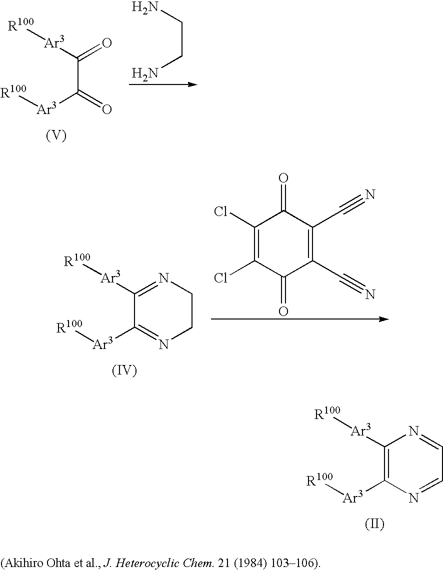

Accordingly, tetrasubstituted pyrazine compounds of the present invention of the formula: 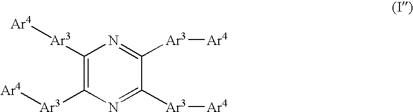
can, for example, be prepared by reacting a compound of formula 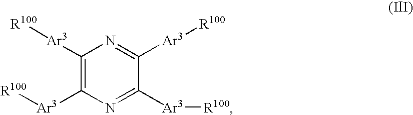
with a boronic acid derivative

E¹-Ar4, or—in case R¹⁰⁰ is not halogen—Hal-Ar4 in the presence of an allylpalladium catalyst of the μ-halo(triisopropylphosphine)(η³-allyl)palladium(II) type.

The intermediates of formula II and III, such as 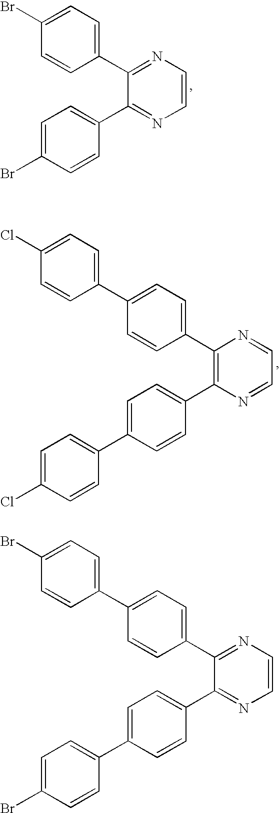
form a further object of the present application.

C₁-C₁₈alkyl is a branched or unbranched radical such as for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, or octadecyl. C₁-C₁₈Alkoxy radicals are straight-chain or branched alkoxy radicals, e.g. methoxy, ethoxy, n-propoxy, isopropoxy, n-butoxy, sec-butoxy, tert-butoxy, amyloxy, isoamyloxy or tert-amyloxy, heptyloxy, octyloxy. isooctyloxy, nonyloxy, decyloxy, undecyloxy, dodecyloxy, tetradecyloxy, pentadecyloxy, hexadecyloxy, heptadecyloxy and octadecyloxy.

C₂-C₁₈Alkenyl radicals are straight-chain or branched alkenyl radicals, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec4-enyl.

C₂-24Alkynyl is straight-chain or branched and preferably C₂-8alkynyl, which may be unsubstituted or substituted, such as, for example, ethynyl, 1-propyn-3-yl, 1-butyn4-yl, 1-pentyn-5-yl, 2-methyl-3-butyn-2-yl, 1,4-pentadiyn-3-yl, 1,3-pentadiyn-5-yl, 1-hexyn6-yl, cis-3-methyl-2-penten-4-yn-1-yl, trans-3-methyl-2-penten-4-yn-1 -yl, 1,3-hexadiyn-5-yl, 1-octyn8-yl, 1-nonyn-9-yl, 1-decyn-10-yl, or 1-tetracosyn-24-yl.

C₄-C₁₈acydoalkyl is preferably C₅-C₁₂cydoalkyl, such as, for example, cyclopentyl, cydohexyl, cydoheptyl, cyclooctyl, cydononyl, cydodecyl, cydododecyl. Cydohexyl and cydododecyl are most preferred.

The term “aryl group” is typically C₈-C₁₈aryl, such as phenyl, indenyl, azulenyl, naphthyl, biphenyl, terphenylyl or quadphenylyl, as-indacenyl, s-indacenyl, acenaphthylenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Examples of COC₁₈aryl are phenyl, 1-naphthyl, 2-naphthyl, 3- or 4-biphenyl, 9-phenanthryl, 2- or 9-fluorenyl, which may be unsubstituted or substituted.

C₇-C₂₄aralkyl radicals are preferably C₇-C₁₈aralkyl radicals, which may be substituted, such as, for example, benzyl, 2-benzyl-2-propyl, β-phenyl-ethyl, α,α-dimethylbenzyl, ω-phenyl-butyl, ω,ω-odimethyl-ωphenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ωphenyl-butyl, ω,ωdimethyl-ω-phenyl-butyl, ω-phenyl-dodecyl or ωphenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted.

C₇-C₁₂alkylaryl is, for example, a phenyl group substituted with one, two or three C₁-C6alkyl groups, such as, for example, 2-, 3-, or 4-methylphenyl, 2-, 3-, or 4 -ethylphenyl, 3-, or 4-isopropylphenyl, 3,4-dimethylphenyl, 3,5-dimethylphenyl, or 3,4,5-trimethylphenyl.

The term “heteroaryl group”, especially C₂-C30heteroaryl, is a ring, wherein nitrogen, oxygen or sulfur are the possible hetero atoms, and is typically an unsaturated heterocydic radical with five to 18 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocydic radicals, which may be unsubstituted or substituted.

Halogen is fluorine, chlorine, bromine and iodine.

Examples of a five or six membered ring formed by R⁵ and R⁶ are heterocydoalkanes or heterocycloalkenes having from 3 to 5 carbon atoms which can have one additional hetero atom selected from nitrogen, oxygen and sulfur, for example 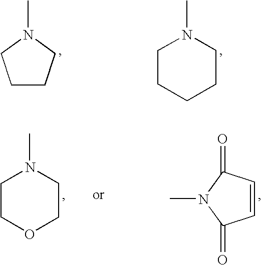
which can be part of a bicyclic system, for example

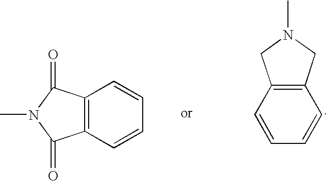

Possible substituents of the above-mentioned groups are C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group or a silyl group.

As described above, the aforementioned radicals may be substituted by E and/or, if desired, interrupted by D. Interruptions are of course possible only in the case of radicals containing at least 2 carbon atoms connected to one another by single bonds; C₆-C₁₈aryl is not interrupted; interrupted arylalkyl or alkylaryl contains the unit D in the alkyl moiety. C₁-C₁₈alkyl substituted by one or more E and/or interrupted by one or more units D is, for example, (CH₂CH₂O)_{1-9—R}^{x}, where R^{x} is H or C₁-C₁₀alkyl or C₂-C₁₀alkanoyl (e.g. CO—CH(C2H₅)C₄H₉), CH2—CH(OR^{Y}′)—CH2—O—R^{y}, where R^{y} is C₁-C₁₈alkyl, C₅-C₁₂cydoalkyl, phenyl, C₇-C₁₅phenylalkyl, and R^{y}′ embraces the same definitions as R^{y} or is H; C₁-C₈alkylene-COO—R^{z}, e.g. CH₂COOR^{z}. CH(CH₃)COOR^{z}, C(CH₃2COOR^{z}, where R^{z} is H, C₁-C₁₈alkyl, (CH₂CH2O)₁₋₉R^{x}, and R^{x} embraces the definitions indicated above; CH₂CH2O—CO—CH═CH₂; CH₂CH(OH)CH2O—CO—C(CH₃)═CH₂.

The electroluminescent devices may be employed for full color display panels in, for example, mobile phones, televisions and personal computer screens.

In general, the pyrazine compound or compounds emit light below about 520 nm, in particular between about 380 nm and about 520 nm.

The pyrazine compound or compounds have a NTSC coordinate of between about (0.12, 0.05) and about (0.16,0.10), preferably a NTSC coordinate of about (0.14, 0.08).

The pyrazine compound or compounds have a melting point above about 150° C., preferably above about 200° C. and most preferred above about 250° C.

To obtain organic layers of this invention with the proper T_{g}, or glass transition temperature, it is advantageous that the present organic compounds have a melting point greater than about 150° C., for example greater than about 200° C., for example greater than about 250° C., for instance greater than about 300° C.

The electroluminescent devices of the present invention are otherwise designed as is known in the art, for example as described in U.S. Pat. Nos. 5,518,824, 6,225,467, 6,280,859, 5,629,389, 5,486,406, 5,104,740, 5,116,708 and 6,057,048, the relevant disclosures of which are hereby incorporated by reference.

For example, organic EL devices contain one or more layers such as: substrate; base electrode; hole-injecting layer; hole transporting layer; emitter layer; electron-transporting layer; electron-injecting layer; top electrode; contacts and encapsulation.

This structure is a general case and may have additional layers or may be simplified by omitting layers so that one layer performs a plurality of tasks. For instance, the simplest organic EL device consists of two electrodes which sandwich an organic layer that performs all functions, including the function of light emission.

A preferred EL device comprises in this order:
(a) an anode,
(b) a hole injecting layer and/or a hole transporting layer,
(c) a light-emitting layer,
(d) optionally an electron transporting layer and
(e) a cathode.

In particular, the present organic compounds function as light emitters and are contained in the light emission layer or form the light-emitting layer.

The light emitting compounds of this invention exhibit intense fluorescence in the solid state and have excellent electric-field-applied light emission characteristics. Further, the light emitting compounds of this invention are excellent in the injection of holes from a metal electrode and the transportation of holes; as well as being excellent in the injection of electrons from a metal electrode and the transportation of electrons. They are effectively used as light emitting materials and may be used in combination with other hole transporting materials, other electron transporting materials or other dopants.

The organic compounds of the present invention form uniform thin films. The light emitting layers may therefore be formed of the present organic compounds alone.

Alternatively, the light-emitting layer may contain a known light-emitfing material, a known dopant, a known hole transporting material or a known electron transporting material as required. In the organic EL device, a decrease in the brightness and life caused by quenching can be prevented by forming it as a multi-layered structure. The light-emitting material, a dopant, a hole-injecting material and an electron-injecting material may be used in combination as required. Further, a dopant can improve the light emission brightness and the light emission efficiency, and can attain the red or blue light emission. Further, each of the hole transporting zone, the light-emitting layer and the electron transporting zone may have the layer structure of at least two layers. In the hole transporting zone in this case, a layer to which holes are injected from an electrode is called “hole-injecting layer”, and a layer which receives holes from the hole-injecting layer and transport the holes to a light-emitting layer is called “hole transporting layer”. In the electron transporting zone, a layer to which electrons are injected from an electrode is called “electron-injecting layer”, and a layer which receives electrons from the electron-injecting layer and transports the electrons to a light-emitting layer is called “electron transporting layer”. These layers are selected and used depending upon factors such as the energy level and heat resistance of materials and adhesion to an organic layer or metal electrode.

The light-emitting material or the dopant which may be used in the light-emitting layer together with the organic compounds of the present invention includes for example anthracene, naphthalene, phenanthrene, pyrene, tetracene, coronene, chrysene, fluorescein, perylene, phthaloperylene, naphthaloperylene, perinone, phthaoperinone, naphthaloperinone, diphenylbutadiene, tetraphenylbutadiene, coumarine, oxadiazole, aldazine, bisbenzoxazoline, bisstyryl, pyrazine, cyclopentadiene, quinoline metal complex, aminoquinoline metal complex, benzoquinoline metal complex, imine, diphenylethylene, vinyl anthracene, diaminocarbazole, pyran, thiopyran, polymethine, merocyanine, an imidazole-chelated oxynoid compound, quinacridone, rubrene, and fluorescent dyestuffs for a dyestuff laser or for brightening.

The pyrazine compounds of the present invention and the above compound or compounds that can be used in a light-emitting layer may be used in any mixing ratio for forming a light-emitting layer. That is, the organic compounds of the present invention may provide a main component for forming a light-emitting layer, or they may be a doping material in another main material, depending upon a combination of the above compounds with the organic compounds of the present invention.

The hole-injecting material is selected from compounds which are capable of transporting holes, are capable of receiving holes from the anode, have an excellent effect of injecting holes to a light-emitting layer or a light-emitting material, prevent the movement of excitons generated in a light-emitting layer to an electron-injecting zone or an electron-injecting material and have the excellent capability of forming a thin film. Suitable hole-injecting materials include for example a phthalocyanine derivative, a naphthalocyanine derivative, a porphyrin derivative, oxazole, oxadiazole, triazole, imidazole, imidazolone, imidazolthione, pyrazoline, pyrazolone, tetrahydroimidazole, oxazole, oxadiazole, hydrazone, acylhydrazone, polyarylalkane, stilbene, butadiene, benzidine type triphenylamine, styrylamine type triphenylamine, diamine type triphenylamine, derivatives of these, and polymer materials such as polyvinylcarbazole, polysilane and an electroconducting polymer.

In the organic EL device of the present invention, the hole-injecting material which is more effective is an aromatic tertiary amine derivative or a phthalocyanine derivative. Although not specially limited, specific examples of the tertiary amine derivative include triphenylamine, tritolylamine, tolyidiphenylamine, N,N′-diphenyl-N,N′-(3-methylphenyl)1,1 -biphenyl4,4′-diamine, N,N,N′,N′-tetra(4-methylphenyl)-1,I′-phenyl-4,4′-diamine, N,N,N′,N′-tetra(4-methylphenyl)-1,1′-biphenyl-4,4′-diamine, N,N′-diphenyl-N,N′-di(1 -naphthyl)-1,1′-biphenyl-4,4′-diamine, N,N′-di(methylphenyl)-N ,N′-di(4-n-butylphenylyphenanthrene-9,10-diamine, 4,4′, 4″-tris(3-methylphenyl)-N-phenylamino)triphenylamine, 1,1-bis(4-di-p-tolylaminophenyl)cyclohexane, and oligomers or polymers having aromatic tertiary amine structures of these.

Although not specially limited, specific examples of the phthalocyanine (Pc) derivative include phthalocyanine derivatives or naphthalocyanine derivatives such as H₂Pc, CuPc, CoPc, NiPc, ZnPc, PdPc, FePc, MnPc, ClAlPc, ClGaPc, ClInPc, ClSnPc, Cl₂SiPc, (HO)AlPc, (HO)GaPc, VOPc, TiOPc, MoOPc, and GaPc—O—GaPc.

The hole transporting layer can reduce the driving voltage of the device and improve the confinement of the injected charge recombination within the pyrazine light emitting layer. Any conventional suitable aromatic amine hole transporting materials described for the hole-injecting layer may be selected for forming this layer.

A preferred class of hole transporting materials is comprised of 4,4′-bis(9-carbazolyl)-1,1′-biphenyl compounds of the formula 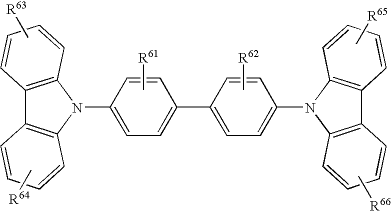
wherein R⁶¹ and R⁶² is a hydrogen atom or a C₁-C₃alkyl group; R⁶³ through R⁶⁶ are substituents independently selected from the group consisting of hydrogen, a C₁-C₈alkyl group, a C₁-Cralkoxy group, a halogen atom, a dialkylamino group, a C₆-C₃₀-aryl group, and the like. Illustrative examples of 4,4′-bis(9-mrbazolyl)-1,1′-biphenyl compounds include 4,4′-bis(9-carbazolyl)-1,1 ′-biphenyl and 4,4′-bis(3-methyl-9-carbazolyl)-1, I′-biphenyl, and the like. The electron transporting layer is not necessarily required for the present device, but is optionally and preferably used for the primary purpose of improving the electron injection characteristics of the EL devices and the emission uniformity. Illustrative examples of electron transporting compounds, which can be utilized in this layer, include the metal chelates of 8-hydroxyquinoline as disdosed in U.S. Pat. Nos. 4,539,507, 5,151,629, and 5,150,006, the disdosures of which are totally incorporated herein by reference. Although not specially limited, specific examples of the metal complex compound include lithium 8-hydroxyquinolinate, zinc bis(8-hydroxyquinolinate), copper bis(8-hydroxyquinolinate), manganese bis(8-hydroxyquinolinate), aluminum tris(8-hydroxyquinolinate), aluminum tris(2-methyl-8-hydroxyquinolinate), gallium tris(8-hydroxyquinolinate), beryllium bis(10-hydroxybenzo[h]quinolinate), zinc bis(10-hydroxybenzo[h]quinolinate), chlorogallium bis(2-methyl-8-quinolinate), gallium bis(2-methyl-8quinolinate)(oaersolate), aluminum bis(2-methyl8-quinolinate)(1-naphtholate), gallium bis(2-methyl-8-quinolinate)(2-naphtholate), gallium bis(2-methyl-8-quinolinate)phenolate, zinc bis(o-(2-benzooxazolyl)phenolate), zinc bis(o-(2-benzothiazolyl)phenolate) and zinc bis(o-(2-benzotrizolyl)phenolate). The nitrogen-containing five-membered derivative is preferably an oxazole, thiazole, thiadiazole, or triazole derivative. Although not specially limited, specific examples of the above nitrogen-containing five-membered derivative include 2,5-bis(1-phenyl)-1,3,4-oxazole, 1,4-bis(2-(4-methyl-5-phenyloxazolyl)benzene, 2,5-bis(1-phenyl)-1,3,4-thiazole, 2,5-bis(1-phenyl)-1,3,4-oxadiazole, 2-(4′-tert-butylphenyl)-5-(4″-biphenyl)1,3,4-oxadiazole, 2,5-bis(1-naphthyl)1,3,4-oxadiazole, 1,4-bis[2-(5-phenyloxadiazolyl)]benzene, 1,4-bis[2-(5-phenyloxadiazolyl)-4-tert-butylbenzene], 2-4′-tert-butylphenyl)5-(4″-biphenyl)-1,3,4-thiadiazole, 2,5-bis(1-naphthyl)-1,3,4-thiadiazole, 1,4-bis[2-(5-phenylthiazolyl)]benzene, 2-(4′-tert-butylphenyl5-(4″-biphonyl)-1,3,4-triazole, 2,5-bis(1-naphthyl)-1,3,4-triazole and 1,4-bis[2-(5-phenyltriazolyl)]benzene. Another class of electron transport materials are oxadiazole metal chelates, such as bis[2-(2-hydroxyphenylI5-phenyl-1,3,4-oxadiazolato]zinc; bis[2-(2-hydroxyphenyl)-5-phenyl-1,3,4-oxadiazolato]beryllium; bis[2-(2-hydroxyphenyl)5-(1-naphthyl)1,3,4-oxadiazolatozinc; bis[2-(2-hydroxyphenyl)-5-(1-naphthyl)-1,3,4-oxadiazolato]beryllium; bis[5-biphenyl-2-(2-hydroxyphenyl)-1,3,4-oxadiazolato]zinc; bis[5-biphenyl-2-(2-hydroxyphenyl)-1,3,4-oxadiazolato]beryllium; bis(2-hydroxyphenyl)-5-phenyl-1,3,4-oxadiazolato]lithium; bis[2-(2-hydroxyphenylI5-p-tolyl-1,3,4-oxadiazolatojzinc; bis 2-(2-hydroxyphenyl)-5-p-tolyl-1,3,4-oxadiazolato]beryllium; bis[5-(p-tert-butylphenylY2-(2-hydroxyphenyl)1,3,4-oxadiazolato]zinc; bis[5-(p-tert-butylphenyl)-2-(2-hydroxyphenyl)-1,3,4-oxadiazolato]beryllium; bis[2-(2-hydroxyphenyl)-5-(3-fluorophenyly 1,3,4-oxadiazolato]zinc; bis[2-(2-hydroxyphenyl)5-(4-fluorophenyl)-1,3,4-oxadiazolato]zinc; bis[2-(2-hydroxyphenyl)-5-(4-fluorophenylyl,3,4-oxadiazolato]beryllium; bis[5-(4-chlorophenyl)-2-(2-hydroxyphenyl)-1,3,4-oxadiazolato]zinc; bis[2-(2-hydroxy phenyl)5-(4-methoxyphenyl)-1,3,4-oxadiazolato]zinc; bis[2,4,2-hydroxy-4-methylphenyl)-5-phenyl-1,3,4-oxadiazolato]zinc; bis[2-.alpha.-(2-hydroxynaphthyl)5-phenyl-1,3,4-oxadiazolato]zinc; bis[2-(2-hydroxyphenyl)5-p-pyridyl-1,3,4-oxadiazolato]zinc; bis[2-(2-hydroxyphenyl)5-p-pyridyl-1,3,4-oxadiazolato]beryllium; bis[2-2-hydroxyphenyl)5-2-thiophenyl)-1,3,4-oxadiazolato]zinc; bis[2-(2-hydroxyphenyl)5-phenyl-1,3,4-thiadiazolato]zinc; bis[2-(2-hydroxyphenyl)5-phenyl-1,3,4-thiadiazolato]beryllium; bis[2-(2-hydroxyphenyly5-(1-naphthyl)-1,3,4-thiadiazolato]zinc; and bis[2-(2-hydroxyphenyl)5-1-naphthyl)1,3,4-thiadiazolato]beryllium, and the like.

In the organic EL device of the present invention, the light-emitting layer may contain, in addition to the light-emitting organic material of the present invention, at least one of other light-emitting material, other dopant, other hole-injecting material and other electron-injecting material. For improving the organic EL device of the present invention in the stability against temperature, humidity and ambient atmosphere, a protective layer may be formed on the surface of the device, or the device as a whole may be sealed with a silicone oil, or the like.

The electrically conductive material used for the anode of the organic EL device is suitably selected from those materials having a work function of greater than 4 eV. The electrically conductive material includes carbon, aluminum, vanadium, iron, cobalt, nickel, tungsten, silver, gold, platinum, palladium, alloys of these, metal oxides such as tin oxide and indium oxide used for ITO substrates or NESA substrates, and organic electroconducting polymers such as polythiophene and polypyrrole.

The electrically conductive material used for the cathode is suitably selected from those having a work function of smaller than 4 eV. The electrically conductive material includes magnesium, calcium, tin, lead, titanium, yttrium, lithium, ruthenium, manganese, aluminum and alloys of these, while the electrically conductive material shall not be limited to these. Examples of the alloys include magnesium/silver, magnesium/indium and lithium/aluminum, while the alloys shall not be limited to these. Each of the anode and the cathode may have a layer structure formed of two layers or more as required.

For the effective light emission of the organic EL device, at least one of the electrodes is desirably sufficiently transparent in the light emission wavelength region of the device. Further, the substrate is desirably transparent as well. The transparent electrode is produced from the above electrically conductive material by a deposition method or a sputtering method such that a predetermined light transmittance is secured. The electrode on the light emission surface side has for instance a light transmittance of at least 10%. The substrate is not specially limited so long as it has adequate mechanical and thermal strength and has transparency. For example, it is selected from glass substrates and substrates of transparent resins such as a polyethylene substrate, a polyethylene terephthalate substrate, a polyether sulfone substrate and a polypropylene substrate.

In the organic EL device of the present invention, each layer can be formed by any one of dry film forming methods such as a vacuum deposition method, a sputtering method, a plasma method and an ion plating method and wet film forming methods such as a spin coating method, a dipping method and a flow coating method. The thickness of each layer is not specially limited, while each layer is required to have a proper thickness. When the layer thickness is too large, inefficiently, a high voltage is required to achieve predetermined emission of light. When the layer thickness is too small, the layer is liable to have a pinhole, etc., so that sufficient light emission brightness is hard to obtain when an electric field is applied. The thickness of each layer is for example in the range of from about 5 nm to about 10 μm, for instance about 10 nm to about 0.2 μm.

In the wet film forming method, a material for forming an intended layer is dissolved or dispersed in a proper solvent such as ethanol, chloroform, tetrahydrofuran and dioxane, and a thin film is formed from the solution or dispersion. The solvent shall not be limited to the above solvents. For improving the film formability and preventing the occurrence of pinholes in any layer, the above solution or dispersion for forming the layer may contain a proper resin and a proper additive. The resin that can be used includes insulating resins such as polystyrene, polycarbonate, polyarylate, polyester, polyamide, polyurethane, polysulfone, polymethyl methacrylate, polymethyl acrylate and cellulose, copolymers of these, photoconductive resins such as poly-N-vinylcarbozole and polysilane, and electroconducting polymers such as polythiophene and polypyrrole. The above additive includes an antioxidant, an ultraviolet absorbent and a plasticizer.

When the light-emitting organic material of the present invention is used in a light-emitting layer of an organic EL device, an organic EL device can be improved in organic EL device characteristics such as light emission efficiency and maximum light emission brightness. Further, the organic EL device of the present invention is remarkably stable against heat and electric current and gives a usable light emission brightness at a low actuation voltage. The problematic deterioration of conventional devices can be remarkably decreased.

The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light.

The material of the present invention can be used in the fields of an organic EL device, an electrophotographic photoreceptor, a photoelectric converter, a solar cell, an image sensor, dye lasers and the like.

The following Examples illustrate the invention, without limiting the scope thereof. In the Examples and throughout this application, the term light emitting material means the present pyrazine compounds.

### EXAMPLES

### Example 1

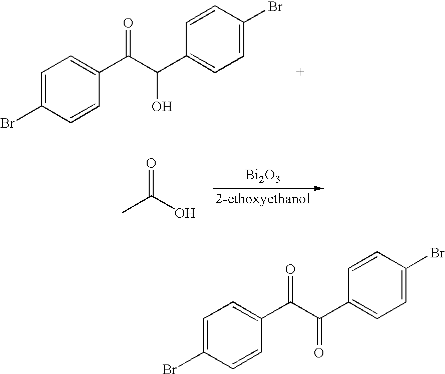 [0151] a) 1,2-Di(4-bromophenyl)-2-hydroxyethanone (0.50 g, 1.4 mmol) is added to 20 ml of 2-ethoxyethanol and 0.3 ml of AcOH. The mixture is heated to 105° C. and then Bi₂O₃ (0.19 g, 0.4 mmol) is added. CH₃-C₁₂ is added to the reaction mixture and an extraction is made with water. The organic phase is washed until the water phase is neutral. The product is redissolved in toluene and filtrated on silica gel. The solvent is evaporated to leave the product as a yellow crystalline material (yield: 0.37 g, 72%, mp. 225-227° C.). ¹H NMR (300 MHz, CDCl₃): δ 7.77 (d, 10.8 Hz, 4H), 7.60 (d, 10.8 Hz, 4H). 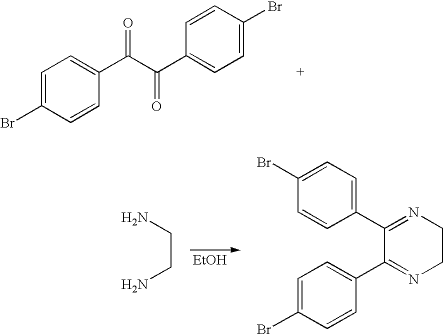
b) First the product obtained in step a) (10.01 g, 27 mmol) is added to 100 ml of ethanol, then ethylene diamine (1.99 g, 33 mmol) is added and the reaction mixture is refluxed for 2 h. During cooling a product precipitates, which is filtered and dried to give a yellow crystalline material (yield: 9.80 g, 92%). ¹H NMR (300 MHz, CDCl₃): δ 7.43 (d, 4.7 Hz, 4H), 7.27 (d, 4.8 Hz, 4H), 3.70 (s, 2H). 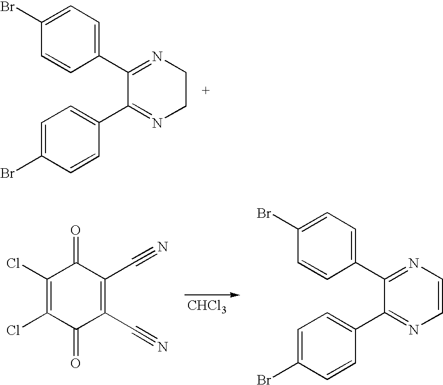
c) The dihydropyrazine (9.02 g, 23 mmol) is dissolved in 50 ml of chloroform and then DDQ (10.44 g, 46 mmol) is added. The reaction mixture is refluxed for 8 h, poured into a NaHCO₃ solution and the water phase is extracted with dichloromethane. The organic phase is washed with NaHCO₃ until the water phase is nearly colourless. The product is purified by column chromatography with dichloromethane as eluant After evaporation, a white solid is obtained (yield: 7.81 g, 87%; mp 153-154 ° C.). ¹H NMR (300 MHz, CDCl₃): δ 8.53 (s, 2H), 7.39 (d, 6.7 Hz, 4H), 7.26 (d, 6.7 Hz, 4H).

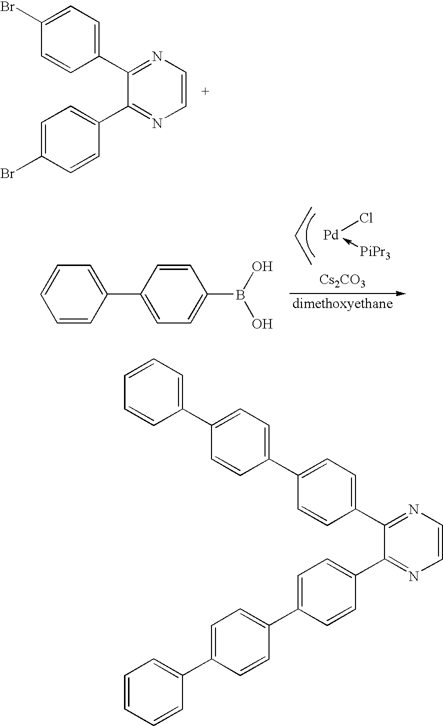

The product obtained in step c) (0.98 g, 2.5 mmol) is added to 50 ml of dimethoxyethane, then biphenylboronic acid (1.24 g, 6.3 mmol) is added and the reaction mixture is stirred under Argon atmosphere for 10 minutes. Cs₂CO₃ (2.04 g, 6.3 mmol) dissolved in 5 ml of water is added. Then the palladium catalysator is added. The reaction mixture is refluxed for 18h. The product is filtered off and recrystallized in DMF to give a grey crystalline material (yield: 0.92 g, 69;%, mp 298-301° C.). ¹H NMR (400 MHz, CDCl₃): δ 8.69 (s, 2H) 7.77-7.68 (m, 20H), 7.51 (t, 7.6 Hz, 4H), 7.43-7.39 (m, 2H).

### Example 2

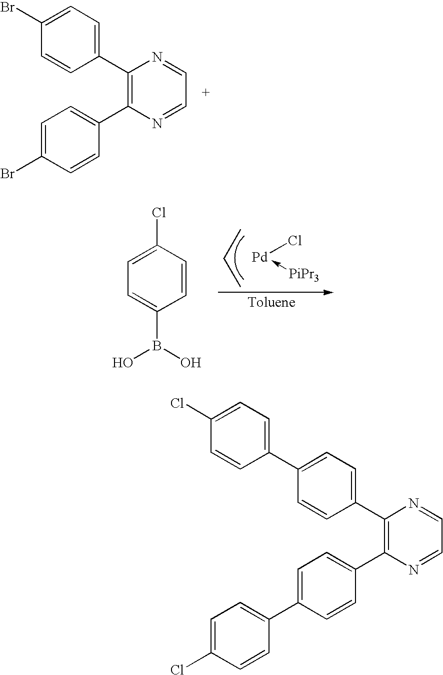

The product obtained in step c) of example 1 (2.00 g, 5.1 mmol) and 4-chlorophenylboronic acid (2.41 g, 15.4 mmol) are added to 100 ml of toluene. The mixture is stirred for 10 minutes under an argon atmosphere. Then Cs₂CO₃ (7.86 g, 24.1 mmol) in 4 ml of water is slowly added to the mixture. After 10 minutes the palladium catalysator is added. The reaction mixture is refluxed for 7 h, CH₃-C₁₂ is added and the solution is extracted with a saturated solution of tartaric acid. The product is recrystallized in ethanol to give a white crystalline material (yield: 1.94 g, 84%, mp 184-185° C.). ¹H NMR (300 MHz, CDCl₃): δ 8.56 (s, 2H), 7.52 (d, 6.6 Hz, 8H), 7.46 (d, 7.8 Hz, 4H), 7.33 (d, 7.0 Hz, 4H).

### Example 3

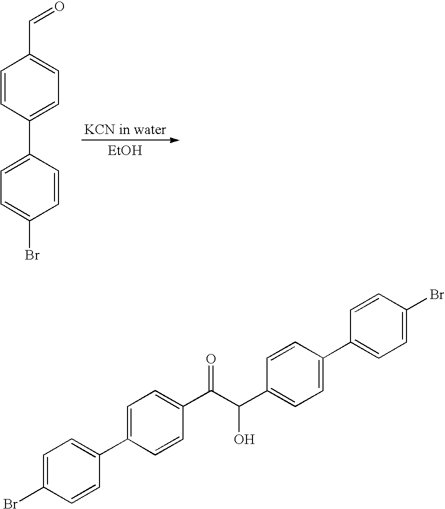 [0158] a) 4-(4′-Bromobiphenyl)methanal (4.18 g, 16 mmol) is added to 10 ml of ethanol. Then KCN (0.03 g, 0.5 mmol) in 5 ml of water is added. The reaction mixture is refluxed. After 90 minutes KCN is added. After 4 h the reaction is finished. The solid is filtered, washed with ethanol, H₂O and ethanol to give a pale yellow solid (yield: 3.39 g (81%), mp. 243-246° C.). ¹H NMR (400 MHz, CDCl₃): δ8.29 (d, 6.7 Hz, 2H), 7.87-7.78 (m, 8H), 7.70-7.64 (m, 6H), 6.27 (d, 6.0 Hz, 1H), 4.84 (d, 6.1 Hz, 1H). 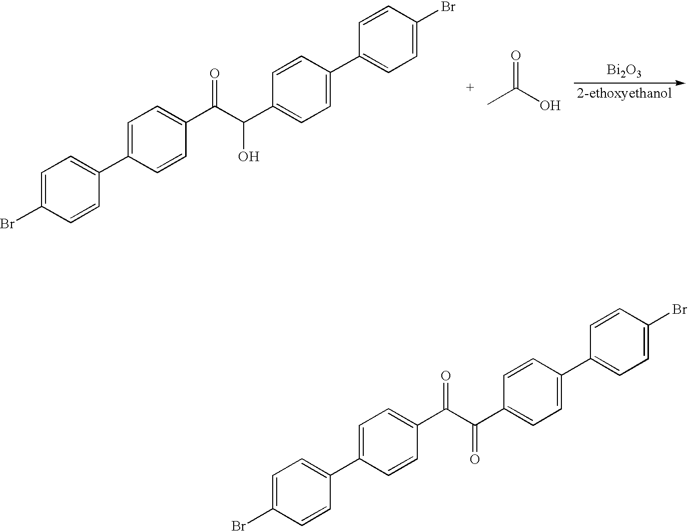
b) The product obtained in step a) (0.80 g, 1.5 mmol) is added to 40 ml of 2-ethoxyethanol and 0.5 ml of ACOH. The mixture is heated at 105° C. and then Bi₂O₃ (0.19 g, 0.4 mmol) is added. After 3 h the reaction is finished. The grey-green product is filtered off (yield: 0.70 g, 88%, mp 259.5-260.5° C.). ¹H NMR (400 MHz, CDCl₃): δ 8.23 (d, 8.0 Hz, 4H), 7.86 (d, 8.4 Hz, 4H), 7.77 (d, 6.8 Hz, 4H), 7.65 (d, 6.8 Hz, 4H). 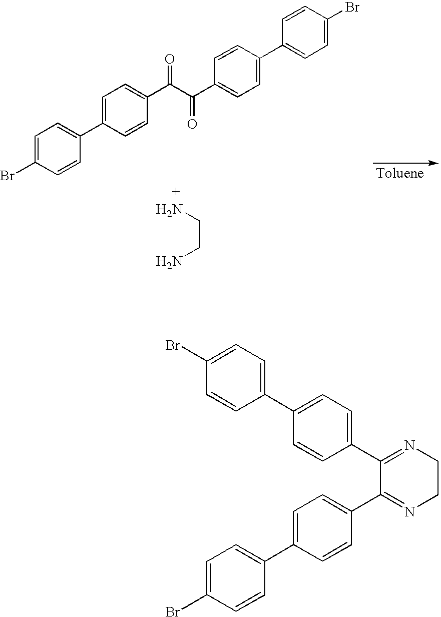
c) the product obtained in step b) (1.80 g, 3.5 mmol) is added to 50 ml of toluene, then ethylene diamine (0.42 g, 6.9 mmol) is added and the reaction mixture is refluxed for 4 h. During cooling a grey crystalline material precipitates, which was filtered off and dried (yield: 1.43 g (76%). ¹H NMR (400 MHz, CDCl₃): δ 7.60-7.44 (m, 16H), 3.76 (s, 4H). 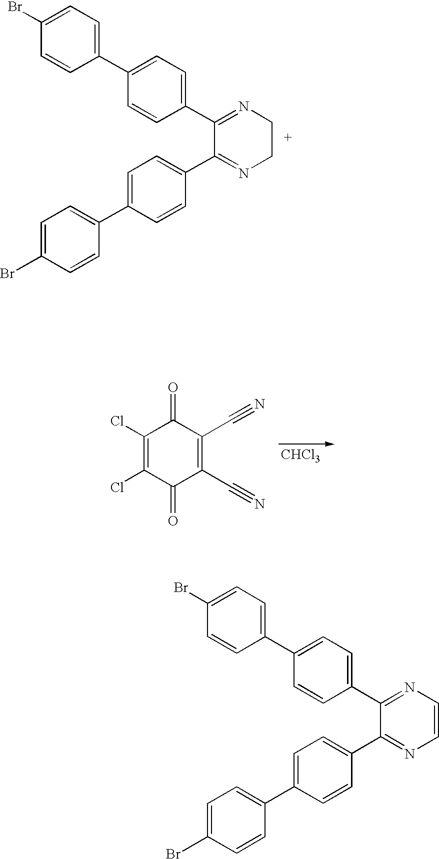
d) The product obtained in step c) (0.33 g, 0.6 mmol) is added to 10 ml of chloroform, and then DDQ (0.27 g, 1.2 mmol) is added. The reaction mixture is refluxed for 4 h. The reaction mixture is poured in a NaHCO₃ solution and the water phase is extracted with dichloromethane. The organic phase is washed with NaHCO₃ until the water phase is nearly colourless. The solvent is removed in vacuum to give a brown-orange solid (yield: 0.3 9 (92 %), mp 214-214.5° C.). ¹H NMR (300 MHz, CDCl₃): δ 8.56 (s, 2H), 7.54-7.38 (m, 16H). 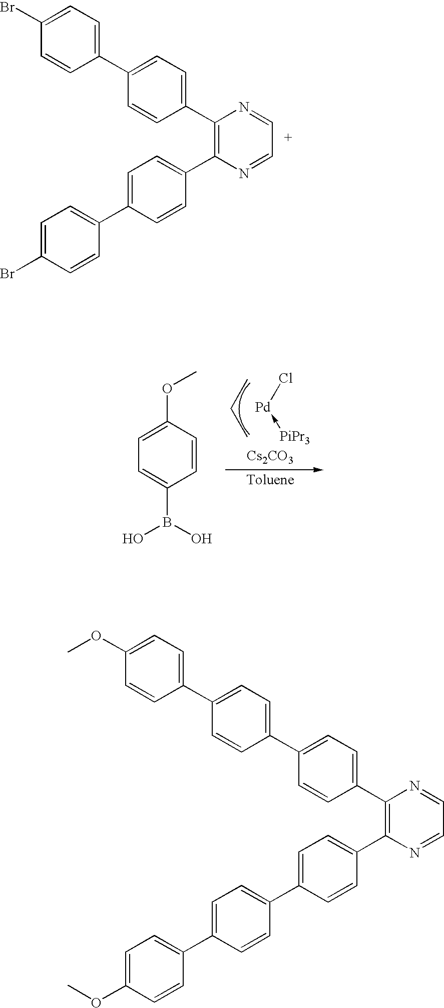
e) The product obtained in step d) (0.80 g, 1.5 mmol) and 4-methoxyphenylboronic acid (0.56 g, 3.7 mmol) are added to 40 ml of toluene. The mixture is stirred for 10 min under an argon atmosphere. Then Cs₂CO₃ (1.41 g, 4.3 mmol) in 5 ml of water is slowly added to the mixture. After 10 minutes palladium catalysator is added. Then the reaction mixture is refluxed for 15 h. The solid phase is filtered off and washed. The product is recrystallized in DMF and then filtered on Hyflo. After solvent evaporation a pale yellow solid remains (yield: 0.20 g (23%, mp. 339.5-341° C.). ¹H NMR (400 MHz, CDCl₃): δ 8.66 (s, 2H), 7.71-7.59 (m, 20H), 7.01 (d, 8.8 Hz, 4H), 3.88 (s, 6H).

### Example 4

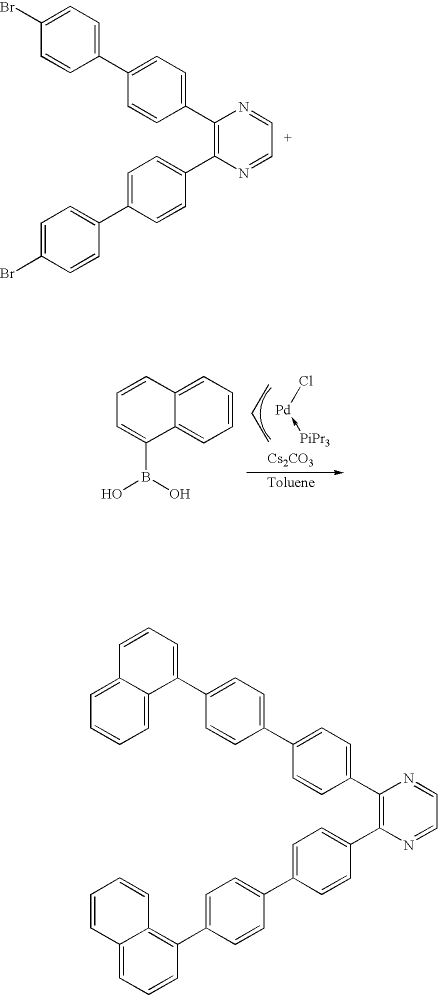

The product obtained in example 3b) (1.20 g, 2.7 mmol) and 1-naphtylboronic add (1.14 g, 6.6 mmol) are added to 50 ml of toluene. The mixture is stirred for 10 minutes under an argon atmosphere. Then Cs₂CO₃ (2.54 g, 7.8 mmol) in 8 ml of water and the palladium catalysator are added. The reaction mixture is refluxed for 18 h. Then the solution is poured into 10% tartaric acid and an extraction is made with dichloromethane. The organic phase is dried with magnesium sulphate and the solvent is removed. Then the crude product was purified by column chromatography on silica gel with dichloromethane. After evaporation a white powder is recovered. Yield: 1.43 g (2.25 mmol) 85%. ¹H NMR (400 MHz, CDCl₃): δ 8.69 (s, 2H), 8.00-7.89 (m, 6H), 7.79 (d, 8.2 Hz, 4H), 7.74-7.72 (m, 8H), 7.69-7.45 (m, 12H).

### Example 5

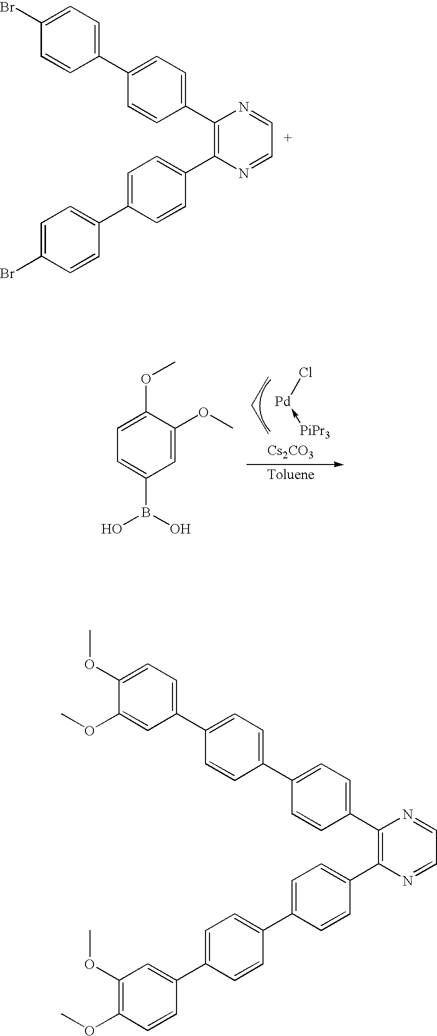

The product obtained in example 3b) (1.20 g, 2.7 mmol) and 3,4dimethoxyphenylboronic acid (1.20 g, 6.6 mmol) are added to 50 ml of toluene. The mixture is stirred for 10 minutes under an argon atmosphere. Then Cs₂CO₃ (2.54 g, 7.8 mmol) in 8 ml of water and the palladium catalysator are added. The reaction mixture is refluxed. After 12 h one equivalent of each reactant is added. The reaction is finished after 18 h. The reaction mixture is then poured into 10% tartaric acid and an extraction is made with dichloromethane. The organic phase is dried with magnesium sulphate and the solvent is removed. The crude product is then dissolved in CH₃-C₁₂ and filtered, wherein a gold crystalline product is obtained (yield: 0.64 g (37%). ¹H NMR (400 MHz, CDCl₃): 8 8.66 (s, 2H), 7.71 (d, 8.4 Hz, 4H), 7.66 (m, 12H), 7.22 (dd, 1.9, 8.3 Hz, 2H), 7.17 (d, 1.9 Hz, 2H), 3.99 (s, 6H), 3.96 (s, 6H).

### Example 6

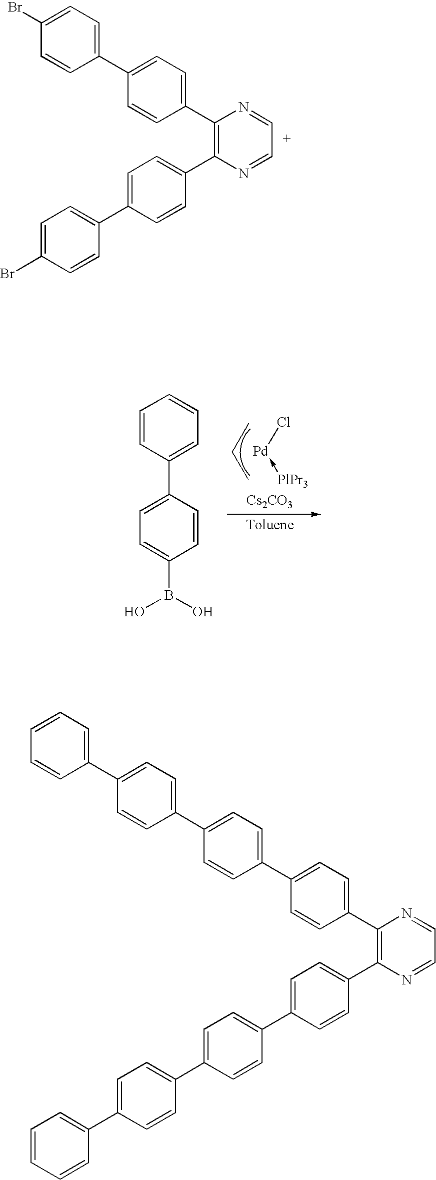

The product obtained in example 4b) (1.40 g, 2.6 mmol) and biphenylboronic acid (1.28 g, 6.5 mmol) are put in 60 ml of toluene. The mixture is stirred for 10 minutes under an argon atmosphere. Then Cs₂CO₃ in 10 ml of water is slowly added to the mixture. After 10 minutes the palladium catalyxsator is added. Then the reaction mixture is refluxed for 72 h. The solid phase is filtered off. The product is recrystallised from isopropanol to obtain a brown solid (yield: 0.20 g (11%)). ¹H NMR (400 MHz, CDCl₃): δ 8.66 (s, 2H), 7.80-4.63 (m, 26H), 7.54-7.36 (m, 8H).

### Application Example (Device)

The following device structure is prepared: ITO/CuPC/TCTA/ Compound of Example 4/TPBI/LiF/Al where ITO is indium tin oxide, CuPC is copper phthalocyanine, TCTA is 4,4′,4″-tri-(N-carbazoyl)triphenylamine, and TPBI is 1,3,5-tris-(N-phenyl-benzimidazol-2-yl) benzene. Using this device structure, a brightness of 106 cd/m² is observed with a efficiency of 0.39 cd/A at 11 V with an emission λₘₐₓ at 450 nm.

1. A pyrazine compound of formula 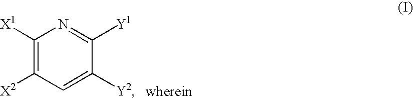
X¹ is a group of formula 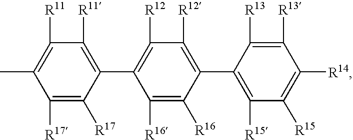
or a C₁₆-C₃₀aryl group, which can optionally be substituted by E;
X² is an aryl group, or a heteroaryl group, which can optionally be substituted;
Y¹ and Y² are independently of each other a hydrogen atom, C₁-C₁₈alkyl, which is optionally interrupted by O,
an aryl group or a heteroaryl group, which can optionally be substituted;
Y¹ and Y² together form a C₅-C₈cycloalkyl group, wherein R¹¹, R^{11′}, R¹², R^{12′}, R¹³, R^{13′}, R¹⁵, R^{15′}, R¹⁶, R^{16′}, R¹⁷ and R^{17′} are independently of each other H, E, C₆-C₁₈aryl; C₆-C₁₈aryl which is substituted by E; C₁-C₁₈alkyl; C₁-C₁₈alkyl which is substituted by E′ and/or interrupted by D; C₇-C₁₈aralkyl; or C₇-C₁₈aralkyl which is substituted by E; or
R^{11′} and R¹², R^{12′} and R¹³, R^{15′} and R¹⁶ and/or R^{16′} and R¹⁷, are each a divalent group L¹ selected from an oxygen atom, an sulfur atom, >CR¹⁸R¹⁹ >SiR¹⁸R¹⁹, or 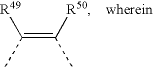
R¹⁸ and R¹⁹ are independently of each other C₁-C₁₈alkyl; C₁-C₁₈alkoxy, C₆-C₁₈aryl, C₆-C₁₈aryl, which is substituted by E; C₇-C₁₈aralkyl, or C₇-C₁₈aralkyl, which is substituted by E; or R¹¹ and R^{11′}, R¹² and R^{12′}, R¹³ and R¹³, R^{13′} and R¹⁴, R^{14′} and R¹⁵, R¹⁵ and R^{15′}, R¹⁶ and R^{16′} and/or R^{17′} and R¹⁷, are each a divalent group 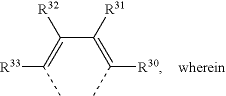
R³⁰, R³¹, R³², R³³, R⁴⁹ and R⁵⁰ are independently of each other H, C₁-C₁₈alkyl; C₁-C₁₈alkyl, which is substituted by E′ and/or interrupted by D; E; C₆-C₁₈aryl; C₆-C₁₈aryl, which is substituted by E; R¹⁴ is H, C₂-C₃₀heteroaryl, —NR⁷⁰R⁷¹, C₆-C₃₀aryl, or C₆-C₃₀aryl which is substituted by E, C₁-C₁₈alkyl; or C₁-C₁₈alkyl which is substituted by E′ and/or interrupted by D; especially
C₇-C₁₈aralkyl; C₇-C₁₈aralkyl which is substituted by E;
wherein R⁷⁰ and R⁷¹ are independently of each other H, C₆-C₁₈aryl, C₆-C₁₈aryl which is substituted by C₁-C₂₄alkyl, or C₁-C₂₄alkoxy; C₁-C₂₄alkyl, or C₁-C₂₄alkyl which is interrupted by —O—, or
R⁷⁰ and R⁷¹ together form a five or six membered ring,
C₁-C₁₈alkyl; C₁-C₁₈alkyl, which is substituted by E and/or interrupted by D; C₂-C₃₀heteroaryl; C₇-C₁₈aralkyl; C₇-C₁₈aralkyl which is substituted by E;
D is —CO—; —COO—; —OCOO—; —S—; —SO—; —SO2—; —O—; —NR⁵—; —SiR⁶¹R⁶²—; —POR⁵—; —CR⁶³═CR⁶⁴—; or —C≡C—;
E is C₁-C₁₈alkyl, —OR⁵; —SR⁵; —NR⁵R⁶; —COR⁸; —COOR⁷; —CONR⁵R⁶; —CN; or halogen;
E′ is E, except C₁-C₁₈alkyl, wherein
R⁵ and R⁶ are independently of each other C₆-C₁₈aryl; C6C₁₈aryl which is substituted by C₁-C₁₈alkyl, or C₁-C₁₈alkoxy; C₁-C₁₈alkyl, or C₁-C₁₈alkyl which is interrupted by —O—; or
R⁵ and R⁶ together form a five or six membered ring,
R⁷ is C₆-C₁₈aryl; C₆-C₁₈aryl which is substituted by C₁-C₁₈alkyl, or C₁-C₁₈alkoxy; C₁-C₁₈alkyl; or
C₁-C₁₈alkyl which is interrupted by -O—;
R⁸ is C₇-C₁₂alkylaryl; C₁-C₁₈alkyl; or C₁-C₁₈alkyl which is interrupted by -O—;
R⁶¹ and R⁶² are independently of each other C₆-C₁₈aryl; C₆-C₁₈aryl which is substituted by C₁-C₁₈alkyl, or C₁-C₁₈alkoxy; C₁-C₁₈alkyl, or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, or C₁-C₁₈alkoxy; C₁-C₁₈alkyl, or C₁-C₁₈alkyl which is interrupted by —O—.

2. A pyrazine compound of formula I according to claim 1, wherein X¹ and X² are independently of each other a group of formula 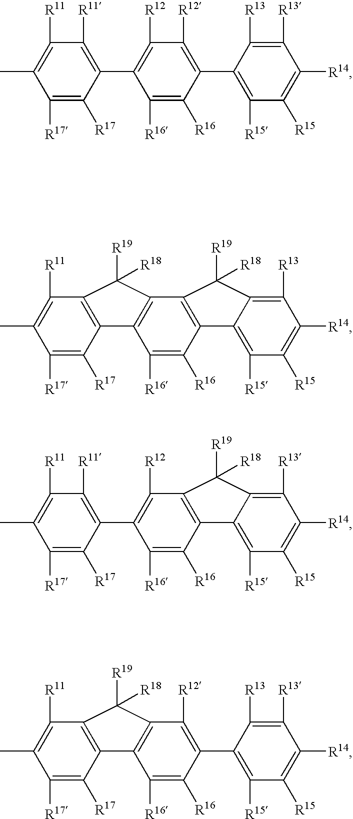
or —X¹¹—X¹²—X¹³, wherein
R¹¹, R^{11′}, R¹², R^{12′}, R¹³, R^{13′}, R¹⁵, R^{15′}, R¹⁶, R^{16′}, R¹⁷ and R^{17′} are independently of each other H, C₆-C₁₈aryl; C₆-C₁₈aryl which is substituted by E; E, C₁-C₁₈alkyl; C₁-C₁₈alkyl which is substituted by E′ and/or interrupted by D; C₇-C₁₈aralkyl; C₇-C₁₈aralkyl which is substituted by E; and X¹¹ and X¹² are independently of each other a group of formula 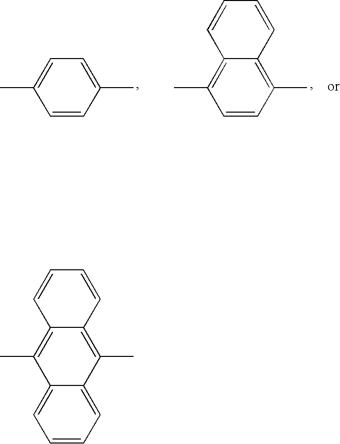
and X¹³ is a group of formula 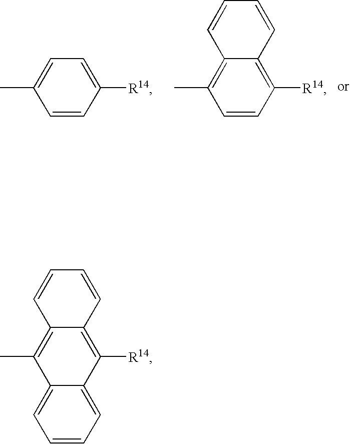
wherein R¹⁴ is 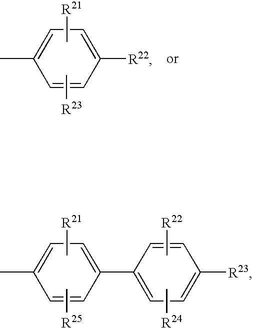
wherein R²¹, R²², R²³, R²⁴ and R²⁵ are independently of each other H, E, C₁-C₁₈alkyl: C₁-C₁₈alkyl which is substituted by E′ and/or interrupted by D and Y¹ and Y² are a hydrogen atom, C₁-C₁₈alkyl, which is optionally interrupted by O, or Y¹ and Y² together form a C₅-C₈cycloalkyl group.

3. The pyrazine compound according to claim 1 wherein R¹¹, R^{11′}, R¹², R^{12′}, R¹³, R^{13′}, R¹⁵, R^{15′}, R¹⁶, R^{16′}, R¹⁷ as well as R¹⁴ are independently of each other H, E; or C₁-C₈alkyl;
wherein E is —OR⁵; —SR⁵; —NR⁵R⁶; —COR⁸; —COOR⁷; —CONR⁵R⁶; —CN; —OCOOR⁷; or halogen;
wherein R⁵ and R⁶ are independently of each other C₆-C₁₂aryl, or C₁-C₈alkyl;
R⁷ is C₇-C₁₂alkylaryl, or C₁-C₈alkyl; and
R⁸ is C₆-C₁₂aryl; or C₁-C₈alkyl; or
R¹¹ and R^{11′}, R¹² and R^{12′}, R¹³ and R^{13′} and/or R^{13′} and R¹⁴ are each a divalent group

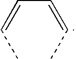

4. The pyrazine compound according to claim 1, wherein
X¹ and X² are a group of formula 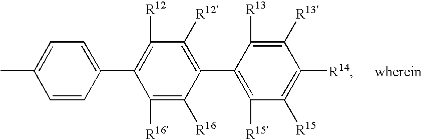
R¹³, R^{13′}, R¹⁵ and R^{15′} are H and R¹⁴ is H, or 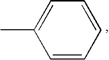
and R¹², R^{12′}, R¹⁶ and R^{16′} are H; or
R¹³ and R¹⁵ are H, R¹³ and R¹⁵ are independently of each other H, C₁-C₈alkyl, or C₁-C₈alkoxy, and R¹⁴ is H, C₁-C₈alkyl, or C₁-C₈alkoxy, and R¹², R^{12′}, R¹⁶ and R^{16′} are H, wherein at least one of R¹³, R¹⁵, R^{13′}, R^{15′} and R¹⁴ is C₁-C₈alkyl, or C₁-C₈alkoxy;
R¹² and R^{12′}, R¹³ and R^{13′} and R¹⁴, R¹⁴ and R¹⁵, R¹⁵ and R^{15′}, and/or R¹⁶ and R^{16′}, can be a divalent group 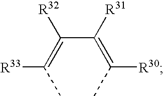
or
R¹², R¹⁶, R^{16′} are H and R¹³ and R^{13′}, or R^{13′} and R¹⁴ and/or R¹⁵ and R^{15′} are a divalent group 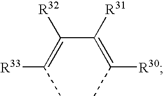
or
R¹³, R^{13′}, R¹⁴, R¹⁵, R^{15′} are H, R¹⁴ is H, C₁-C₈alkyl and R¹² and R^{12′}, and/or R¹⁶ and R¹⁶′ are a divalent group 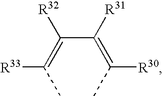
wherein R³⁰, R³¹, R³² and R³³ are H, C₁-C₈alkyl, or C₁-C₈alkoxy, and
Y¹ and Y² are a hydrogen atom.

5. The pyrazine compound according to claim 1, wherein X¹ and X² are independently of each other a group of formula 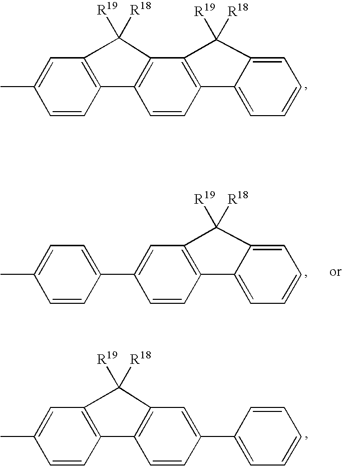
wherein
R¹⁸ and R¹⁹ are independently of each other C₁-C₈alkyl.

6. (canceled)

7. An electroluminescent device, comprising a pyrazine compound of formula I according to claim 1.

8. The electroluminescent device according to claim 7, wherein the electroluminescent device comprises in this order
(a) an anode
(b) a hole injecting layer and/or a hole transporting layer
(c) a light-emitting layer
(d) optionally an electron transporting layer and
(e) a cathode.

9. The electroluminescent device according to claim 8, wherein the pyrazine compound of formula I forms the light-emitting layer.

10. An electrophotographic photoreceptor, photoelectric converter, solar cell, image sensor or dye laser compound of formula I according to claim 1.

11. A pyrazine compound according to claim 1 of formula 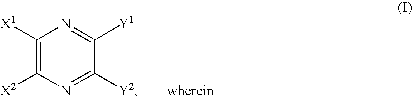
X² is a group of formula 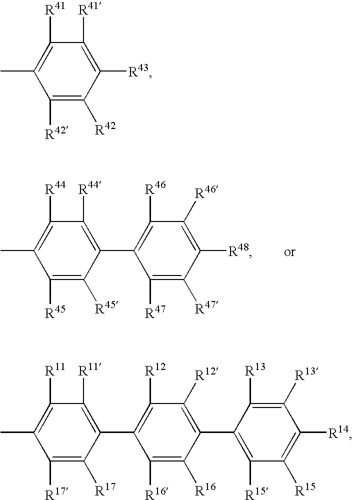
or a C₁₆-C₃₀aryl group, which can optionally be substituted by E;
Y¹ and Y² are independently of each other a hydrogen atom, C₁-C₁₈alkyl, which is optionally interrupted by O,
a C₁₆-C₃₀aryl group, which can optionally be substituted by E; or a group of formula 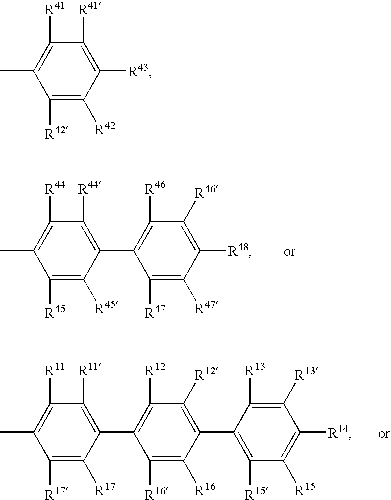
Y¹ and Y² together form a C5-C₈cycloalkyl group, wherein
R⁴¹, R^{41′}, R⁴², R⁴²′, R⁴⁴ R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by E; C₁-C₁₈alkyl; C₁-C₁₈alkyl which is substituted by E′ and/or interrupted by D; C₇-C₁₈aralkyl; or C₇-C₁₈aralkyl which is substituted by E; or R⁴⁴ and R⁴⁶ and/or R^{45′} and R⁴⁷ are each a divalent group L¹ selected from an oxygen atom, an sulfur atom, >CR¹⁸R¹⁹>SiR¹⁸R¹⁹, or 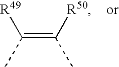
R⁴¹and R^{41′}, R⁴² and R^{42′}, R^{42′} and R⁴³, R^{41′} and R⁴³, R⁴⁴ and R⁴⁴, R⁴⁵ and R⁴⁵, R⁴⁶ and R^{46′}, R⁴⁷ and R⁴⁷′, R⁴⁶ and R⁴⁸ and/or R^{47′} and R⁴⁸ are each a divalent group 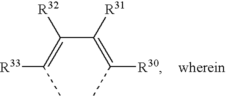
R³⁰, R³¹, R³², R³³, R⁴⁹ and R⁵⁰ are independently of each other H, C₁-C₁₈alkyl; C₁-C₁₈alkyl, which is substituted by E′ and/or interrupted by D; E; C₆-C₁₈aryl; C₆-C₁₈aryl, which is substituted by E; R¹⁴ is H, C₂-C₃₀heteroaryl, —NR⁷⁰R⁷¹, C₆-C₃₀aryl, or C₆-C₃₀aryl which is substituted by E, C₁-C₁₈alkyl; or C₆-C₁₈alkyl which is substituted by E′ and/or interrupted by D;
R⁴³ and R⁴⁸ are independently of each other H, E, or —NR⁷⁰R⁷¹.

12. The pyrazine compound according to claim 2, wherein R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R^{44′}, R⁴⁵, R^{45′}, R⁴⁶, R^{46′}, R⁴⁷ and R^{47′} as well as R¹⁴, R⁴³, and R⁴⁸ are independently of each other H, E; or C₁-C₈alkyl; wherein E is —OR⁵; —SR⁵;
—NR⁵R⁶; —COR⁸; —COOR⁷; —CONR⁵R⁶; —CN; —OCOOR⁷; or halogen; wherein R⁵ and R⁶ are independently of each other C₆-C₁₂aryl, or C₁-C₈alkyl;
R⁷ is C₇-C₁₂alkylaryl, or C₁-C₈alkyl; and R⁸ is C₆-C₁₂aryl; or C₁-C₈alkyl; or
R¹¹ and R^{11′}, R¹² and R^{12′}, R¹³ and R^{13′}, R^{13′} and R¹⁴, R⁴¹ and R^{41′}, R^{41′} and R⁴³, R⁴⁴ and R^{44′}, R⁴⁶ and R^{46′}, R^{46′} and R⁴⁸ and/or R^{47′} and R⁴⁸ are each a divalent group

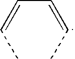

13. The pyrazine compound according to claim 11, wherein
X¹ is a group of formula 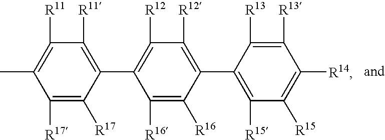
X² is a group of formula 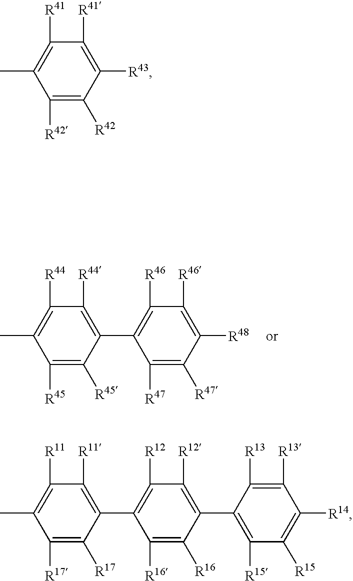
and Y¹ and Y² are a hydrogen atom.

14. The pyrazine compound according to claim 13, wherein R¹¹, R^{1′}, R¹², R^{12′}, R¹³, R^{13′}, R¹⁴, R¹⁵, R^{15′}, R¹⁶, R^{16′} , R¹⁷, R^{17′}, R⁴¹, R^{41′}, R⁴², R^{42′}, R⁴⁴, R^{44′}, R⁴⁵, R^{45′}, R⁴⁶, R⁴⁶′, R⁴⁷, R^{47′}, R⁴³ and R⁴⁸ are
H, C₁-C₈alkyl, C₁-C₈alkoxy, or phenyl, or R¹³ and R^{13′}, R^{13 ′} and R¹⁴, R¹⁴ and R¹⁵, or R¹⁵ and R^{15′} can be a divalent group

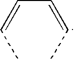

15. The pyrazine compound according to claim 13, wherein 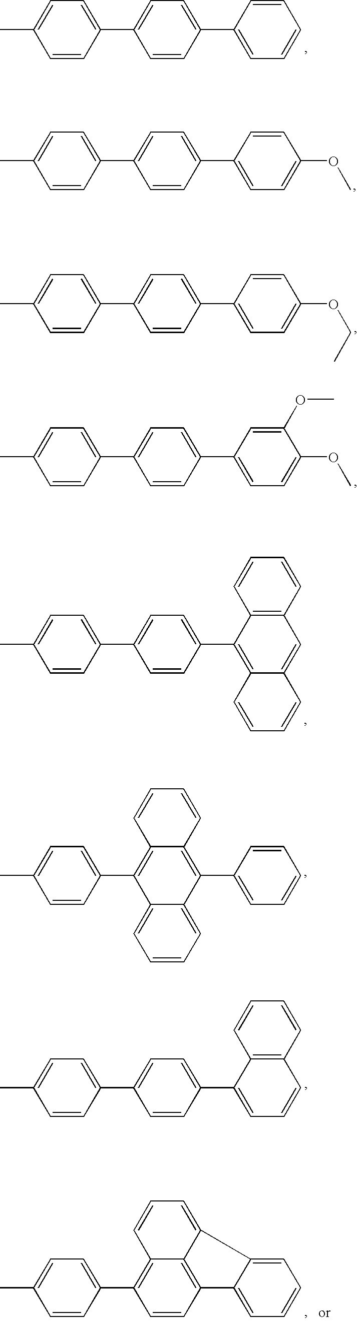 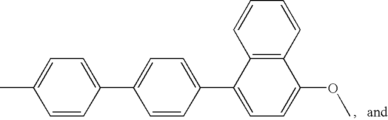
X¹ is
X² is

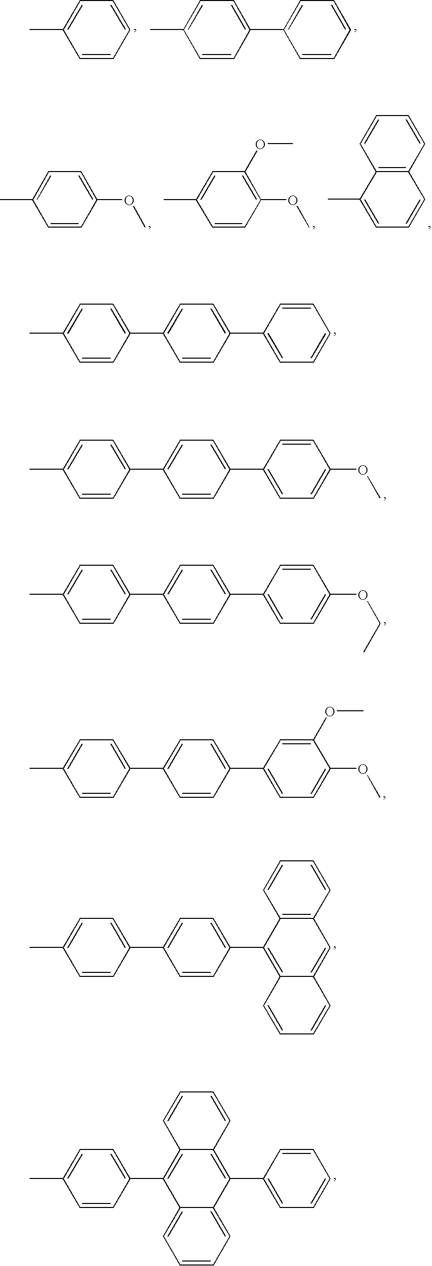

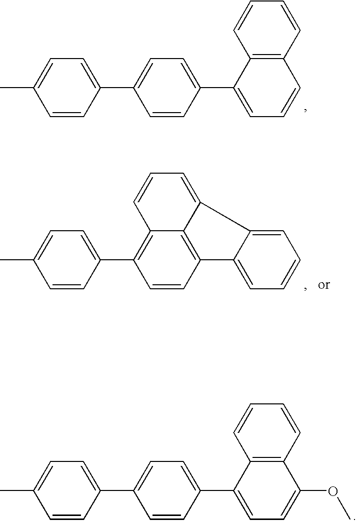

16. The pyrazine compound according to claim 11, wherein
Y¹ and Y² are hydrogen and X¹ and X² are independently of each other a group Ar¹-Ar², wherein
Ar¹ is a group of formula 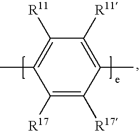
Ar² is a group of formula 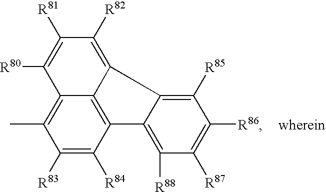
R⁸⁰, R⁸¹, R^{82, R}⁸³, R⁸⁴, R⁸⁵, R⁸⁶, R⁸⁷ and R⁸⁸ are independently of each other H, E′, C₆-C18aryl; C₆-C₁₈aryl, which is substituted by E; C₁-C₁₈alkyl; C₁-C₁₈alkyl which is substituted by E′ and/or interrupted by D; C₇-C₁₈aralkyl; or C₇-C₁₈aralkyl which is substituted by E; e is an integer 1 or 2; or
Y¹ and Y² are independently of each other hydrogen or a group of the formula —W¹ —(W²)_{b}—W³, wherein b is 0 or 1, and
X¹ and X² are independently of each other a group —W¹ —(W²)_{b}—W³, wherein
W¹ and W² are independently of each other a group of formula 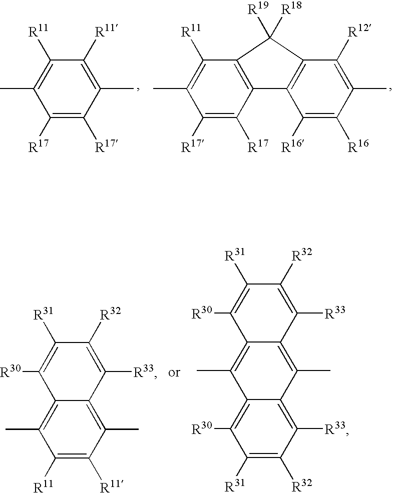
W³ is a group of formula 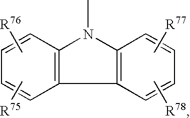
or —NR⁷⁰R⁷¹, wherein R⁷⁰ and R⁷¹ are independently of each other a group of formula 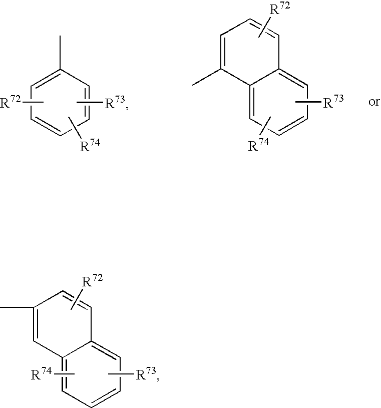
wherein R⁷², R⁷³ and R⁷⁴ are independently of each other hydrogen, C₁-C₈alkyl, C₁-C₈alkoxy, C₁-C₈alkylthio, a cyano group, a carbamoyl group, an amino group, a silyl group or a siloxanyl group,
R⁷⁵, R⁷⁶, R⁷⁷ and R⁷⁸ are independently of each other H, E, C₆-C₁₈aryl; C₆-C₁₈aryl which is substituted by C₁-C₁₈alkoxy, C₁-C₁₈alkyl, C₁-C₁₈alkyl which is interrupted by —O—; C₇-C₁₈aralkyl; or C₇-C₁₈aralkyl which is substituted by C₁-C₁₈alkoxy.
